(19) United States
(12) Patent Application Publication
(10) Pub. No.: US 2022/0356170 A1
(43) Pub. Date: Nov. 10, 2022

(54) SREBP INHIBITOR COMPRISING A THIOPHENE CENTRAL RING

(71) Applicant: Capulus Therapeutics, LLC, San Francisco, CA (US)

(72) Inventors: Michael John GREEN, Half Moon Bay, CA (US); Barry Patrick HART, Palo Alto, CA (US)

(21) Appl. No.: 17/743,235

(22) Filed: May 12, 2022

Related U.S. Application Data
(63) Continuation of application No. PCT/US2020/060276, filed on Nov. 12, 2020.
(60) Provisional application No. 62/935,028, filed on Nov. 13, 2019.
(60) Provisional application No. 62/966,356, filed on Jan. 27, 2020.
(60) Provisional application No. 63/056,408, filed on Jul. 24, 2020.

(51) Int. Cl. C07D 401/14; A61P 35/00

(52) U.S. Cl. CPC C07D 401/14; A61P 35/00

(57) ABSTRACT

Provided herein is the compound (3-chloro-4-(4-(2-(2-hydroxypropan-2-yl)pyridin-4-yl)thiophen-2-yl)phenyl)(4-hydroxypiperidin-1-yl)methanone (Compound 1), and pharmaceutically acceptable salts, solvates, tautomers, isotopes, or isomers thereof. Also provided herein are methods of inhibiting a component of the sterol regulatory element binding protein (SREBP) pathway, such as an SREBP or SREBP cleavage activating protein (SCAP), using Compound 1, or a pharmaceutically acceptable salt, solvate, tautomer, isotope, or isomer thereof. Further provided are methods of treating a disorder in a subject in need thereof, such as liver disease, non-alcoholic steatohepatitis, insulin resistance, or cancer.

### CROSS-REFERENCE TO RELATED APPLICATIONS

This application claims the benefit of U.S. Provisional Application No. 62/935,028, filed Nov. 13, 2019; U.S. Provisional Application No. 62/966,356, filed Jan. 27, 2020; and U.S. Provisional Application No. 63/056,408, filed Jul. 24, 2020, the disclosures of which are hereby incorporated by reference in their entireties.

### FIELD OF THE INVENTION

The present disclosure relates to the compound (3-chloro-4-(4-(2-(2-hydroxypropan-2-yl)pyridin-4-yl)thiophen-2-yl)phenyl)(4-hydroxypiperidin-1-yl)methanone, or a pharmaceutically acceptable salt, solvate, tautomer, isotope, or isomer thereof, their use for inhibiting components of the sterol regulatory element binding protein (SREBP) pathway, such as SREBP or SREBP cleavage activating protein (SCAP), and their use in therapeutic methods of treating disorders.

### BACKGROUND

SREBPs are membrane-bound transcription factors that regulate cholesterol, fatty acid, and triglyceride biosynthesis, and lipid uptake. Fatty acids and lipids are a source of energy and important components of many biological structures, such as lipid membranes of cells. Cholesterol is an important component of biological processes and structures. In mammals, there are three known SREBP isoforms: SREBP-1a, SREBP-1c, and SREBP-2. SREBP-1a controls a broad range of target genes that are involved in the production of fatty acids, triglycerides, phospholipids, and cholesterol. SREBP-1c primarily activates genes which control fatty acid and triglyceride synthesis. SREBP-2 activates genes involved in the synthesis of regulators of cholesterol metabolism, which has been demonstrated in mouse, human, and Drosophila studies. The activity of SREBPs is regulated by SREBP cleavage activating protein (SCAP), which transports SREBP(s) from the endoplasmic reticulum to the Golgi apparatus where the SREBP(s) are proteolytically cleaved, releasing the transcription factor domain.

The pathways regulated by SREBPs and SCAP have been implicated in disorders of metabolism, such as hypertension, dyslipidemia, obesity, type 2 diabetes, insulin resistance, fatty liver, and nonalcoholic steatohepatitis (NASH). NASH, for example, is liver inflammation and hepatocyte ballooning as a result of fat building up in the liver, which can lead to liver damage, such as cirrhosis. NASH can also be associated with other metabolism disorders, such as insulin resistance and metabolic syndrome.

The metabolism of fatty acids, cholesterol, and triglycerides may also be linked to hyperproliferative disorders, such as cancer. One characteristic of the oncogenic transformation of cancer cells is the shift of metabolism from catabolic to anabolic processes. Many cancers require synthesis of fatty acids and other lipids (such as cholesterol), and steroids (such as androgens). Thus, components of the SREBP pathway may play a role in hyperproliferative disorders, such as prostate cancer. SREBP-1c is the major transcriptional regulator of the biosynthesis of fatty acids, and expression of this transcription factor can be stimulated by androgens and epidermal growth factor in prostate cancer cells. Overexpression of SREBP-1c may drive tumorgenicity and invasion of prostate cancer cells. In addition to regulating androgen synthesis, SREBP-2 itself is also regulated by androgens in a direct feedback circuit of androgen production. However, prostate cancer cells have dysfunctional cholesterol homeostasis, resulting in accumulation of cholesterol and increased proliferation. This increase in cholesterol levels has been shown to be driven by regulated by increased SREBP-2 activity. SREBP-2 expression increases during disease progression, and is significantly higher after castration compared to prior.

Regulating components of the SREBP pathway, such as SCAP or SREBPs, is an important therapeutic approach for treating disorders, such as metabolic diseases and cancer. Thus, there is a need for compounds that can inhibit components of the SREBP pathway, such as SREBPs and SCAP.

### BRIEF SUMMARY

In some embodiments, provided herein is (3-chloro-4-(4-(2-(2-hydroxypropan-2-yl)pyridin-4-yl)thiophen-2-yl)phenyl)(4-hydroxypiperidin-1-yl)methanone:

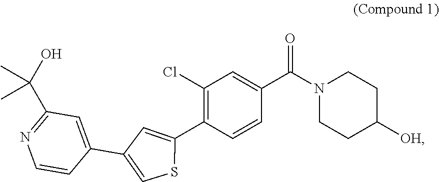 [SMILES: CC(C)(O)c1cc(-c2csc(-c3ccc(C(=O)N4CCC(O)CC4)cc3Cl)c2)ccn1]

or a pharmaceutically acceptable salt, solvate, tautomer, isotope, or isomer thereof.

In other embodiments, provided herein is a pharmaceutical composition which comprises Compound 1, or a pharmaceutically acceptable salt, solvate, tautomer, isotope, or isomer thereof, and a pharmaceutically acceptable excipient.

In yet other embodiments, provided herein is a method of inhibiting a sterol regulatory element-binding protein (SREBP) by contacting the SREBP or contacting an SREBP cleavage activating-protein (SCAP) with Compound 1, or a pharmaceutically acceptable salt, solvate, tautomer, isotope, or isomer thereof; or a pharmaceutical composition comprising any of the foregoing and a pharmaceutically acceptable excipient. In some embodiments, the SREBP is inhibited in a subject in need thereof.

In certain embodiments, provided herein is a method of inhibiting the proteolytic activation of a sterol regulatory element-binding protein (SREBP) by contacting an SREBP cleavage activating-protein (SCAP) with Compound 1, or a pharmaceutically acceptable salt, solvate, tautomer, isotope, or isomer thereof; or a pharmaceutical composition comprising any of the foregoing and a pharmaceutically acceptable excipient. In some embodiments, the proteolytic activation is inhibited in a subject in need thereof.

In other embodiments, provided herein is a method of treating a disorder in a subject in need thereof, wherein the disorder is mediated by a sterol regulatory element-binding protein (SREBP), by administering to the subject an effective amount of Compound 1, or a pharmaceutically acceptable salt, solvate, tautomer, isotope, or isomer thereof; or a pharmaceutical composition comprising any of the foregoing and a pharmaceutically acceptable excipient.

In other embodiments, provided herein is a method of treating a disorder in a subject in need thereof by administering to the subject an effective amount Compound 1, or a pharmaceutically acceptable salt, solvate, tautomer, isotope, or isomer thereof; or a pharmaceutical composition comprising any of the foregoing and a pharmaceutically acceptable excipient.

In certain embodiments, provided herein is the use of Compound 1, or a pharmaceutically acceptable salt, solvate, tautomer, isotope, or isomer thereof, in the manufacture of a medicament for inhibiting a sterol regulatory element-binding protein (SREBP) in a subject in need thereof.

In some embodiments, provided herein is the use of Compound 1, or a pharmaceutically acceptable salt, solvate, tautomer, isotope, or isomer thereof, in the manufacture of a medicament for inhibiting the proteolytic activation of a sterol regulatory element-binding protein (SREBP) in a subject in need thereof.

In some embodiments, provided herein is a method for inhibiting the proteolytic activation of a sterol regulatory element-binding protein (SREBP) in a subject in need thereof, by administering to the subject a therapeutically effective amount of Compound 1, or a pharmaceutically acceptable salt, solvate, tautomer, isotope, or isomer thereof, or a pharmaceutical composition comprising any of the foregoing and a pharmaceutically acceptable excipient.

In some embodiments, provided herein is the use of Compound 1, or a pharmaceutically acceptable salt, solvate, tautomer, isotope, or isomer thereof, for inhibiting the proteolytic activation of a sterol regulatory element-binding protein (SREBP) in a subject in need thereof.

In certain embodiments, provided herein is the use of Compound 1, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

In other embodiments, provided herein is the use of Compound 1, or a pharmaceutically acceptable salt, solvate, tautomer, isotope, or isomer thereof, in the manufacture of a medicament for treating a disorder in a subject in need thereof.

In other embodiments, provided herein is the use of Compound 1, or a pharmaceutically acceptable salt, solvate, tautomer, isotope, or isomer thereof, for inhibiting a sterol regulatory element-binding protein (SREBP). In some embodiments, the SREBP is inhibited in a subject in need thereof.

In certain embodiments, provided herein is the use of Compound 1, or a pharmaceutically acceptable salt, solvate, tautomer, isotope, or isomer thereof, for inhibiting the proteolytic activation of a sterol regulatory element-binding protein (SREBP). In some embodiments, the proteolytic activation is inhibited in a subject in need thereof.

In some embodiments, provided herein is the use of Compound 1, or a pharmaceutically acceptable salt, solvate, tautomer, isotope, or isomer thereof, for treating a disorder in a subject in need thereof, wherein the disorder is mediated by a sterol regulatory element-binding protein (SREBP).

In some embodiments, provided herein is the use of Compound 1, or a pharmaceutically acceptable salt, solvate, tautomer, isotope, or isomer thereof, for treating a disorder in a subject in need thereof.

In some variations of the embodiments described herein, the SREBP is an SREBP-1. In certain variations, the SREBP is SREBP-1a. In other variations, the SREBP is SREBP-1c. In still further embodiments, the SREBP is SREBP-2. In some variations, the disorder is Metabolic Syndrome, type 2 diabetes, obesity, fatty liver disease, insulin resistance, adiposopathy, or dyslipidemia. In other variations, the disorder is a hyperproliferative disorder, such as cancer. In still further variations, the disorder is endotoxic shock, systemic inflammation, or atherosclerosis.

In other embodiments, provided herein is a method of treating fatty liver disease in a subject in need thereof, by administering to the subject an effective amount of Compound 1, or a pharmaceutically acceptable salt, solvate, tautomer, isotope, or isomer thereof; or a pharmaceutical composition comprising any of the foregoing and a pharmaceutically acceptable excipient.

In still further embodiments, provided herein is the use of Compound 1, or a pharmaceutically acceptable salt, solvate, tautomer, isotope, or isomer thereof; or a pharmaceutical composition comprising any of the foregoing and a pharmaceutically acceptable excipient, for treating fatty liver disease in a subject in need thereof.

In yet other embodiments, provided herein is the use of Compound 1, or a pharmaceutically acceptable salt, solvate, tautomer, isotope, or isomer thereof; or a pharmaceutical composition comprising any of the foregoing and a pharmaceutically acceptable excipient, in the manufacture of a medicament for treating fatty liver disease in a subject in need thereof.

In some embodiments, provided herein is a method of treating non-alcoholic steatohepatitis (NASH) in a subject in need thereof, by administering to the subject an effective amount of Compound 1, or a pharmaceutically acceptable salt, solvate, tautomer, isotope, or isomer thereof; or a pharmaceutical composition comprising any of the foregoing and a pharmaceutically acceptable excipient.

In other embodiments, provided herein is the use of Compound 1, or a pharmaceutically acceptable salt, solvate, tautomer, isotope, or isomer thereof; or a pharmaceutical composition comprising any of the foregoing and a pharmaceutically acceptable excipient, for treating non-alcoholic steatohepatitis (NASH) in a subject in need thereof.

In certain embodiments, provided herein is the use of Compound 1, or a pharmaceutically acceptable salt, solvate, tautomer, isotope, or isomer thereof; or a pharmaceutical composition comprising any of the foregoing and a pharmaceutically acceptable excipient, in the manufacture of a medicament for treating non-alcoholic steatohepatitis (NASH) in a subject in need thereof.

In some embodiments, provided herein is a method of treating a hyperproliferative disorder in a subject in need thereof, by administering to the subject an effective amount of Compound 1, or a pharmaceutically acceptable salt, solvate, tautomer, isotope, or isomer thereof; or a pharmaceutical composition comprising any of the foregoing and a pharmaceutically acceptable excipient.

In other embodiments, provided herein is the use of Compound 1, or a pharmaceutically acceptable salt, solvate, tautomer, isotope, or isomer thereof; or a pharmaceutical composition comprising any of the foregoing and a pharmaceutically acceptable excipient, for treating a hyperproliferative disorder in a subject in need thereof.

In certain embodiments, provided herein is the use of Compound 1, or a pharmaceutically acceptable salt, solvate, tautomer, isotope, or isomer thereof; or a pharmaceutical composition comprising any of the foregoing and a pharmaceutically acceptable excipient, in the manufacture of a medicament for treating a hyperproliferative disorder in a subject in need thereof.

### DESCRIPTION OF THE FIGURES

The present application can be understood by reference to the following description taken in conjunction with the accompanying figures.

FIG. 1 is a graph of mean (±SD) plasma concentration vs. time profile of Compound 1 following intravenous administration at 2 mg/kg to male C57 BL/6 mice.

FIG. 2 is a graph of mean (±SD) plasma concentration vs. time profile of Compound 1 following oral administration at 10 mg/kg to male C57 BL/6 mice.

FIG. 3 is a graph of mean (±SD) plasma concentration vs. time profile of Compound 2 following intravenous administration at 2 mg/kg to male C57 BL/6 mice.

FIG. 4 is a graph of mean (±SD) plasma concentration vs. time profile of Compound 2 following oral administration at 10 mg/kg to male C57 BL/6 mice.

FIG. 5 is a graph of tumor volume vs. time profile of Compound 1 following administration of Compound 1 and vehicle in a model with a C33A Endometrial Cell line.

FIG. 6 is a graph of tumor volume vs. time profile of Compound 1 following administration of Compound 1 and vehicle in a model with an A1780 Ovarian Carcinoma Cell line.

### DETAILED DESCRIPTION

### I. Compound 1

Provided herein is the compound (3-chloro-4-(4-(2-(2-hydroxypropan-2-yl)pyridin-4-yl)thiophen-2-yl)phenyl)(4-hydroxypiperidin-1- yl)methanone:

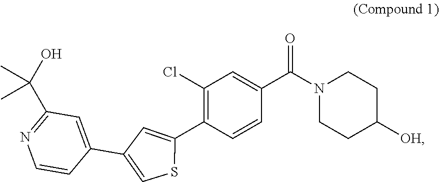 [SMILES: CC(C)(O)c1cc(-c2csc(-c3ccc(C(=O)N4CCC(O)CC4)cc3Cl)c2)ccn1]

or a pharmaceutically acceptable salt, solvate, tautomer, isotope, or isomer thereof.

In some embodiments, provided is a pharmaceutically acceptable salt of Compound 1, or a solvate, tautomer, isotope, or isomer thereof. “Pharmaceutically acceptable salt” includes a salt which is generally safe, non-toxic and not biologically or otherwise undesirable, and includes that which is acceptable for veterinary use as well as human pharmaceutical use. Such salts may include acid addition salts and base addition salts. Acid addition salts may be formed with inorganic acid such as, but not limited to, hydrochloric acid, hydrobromic acid, sulfuric acid, nitric acid, phosphoric acid and the like; or an organic acid such as, but not limited to, acetic acid, 2,2-dichloroacetic acid, adipic acid, alginic acid, ascorbic acid, aspartic acid, benzenesulfonic acid, benzoic acid, 4-acetamidobenzoic acid, camphoric acid, camphor-10-sulfonic acid, capric acid, caproic acid, caprylic acid, carbonic acid, cinnamic acid, citric acid, cyclamic acid, dodecylsulfuric acid, ethane-1,2-disulfonic acid, ethanesulfonic acid, 2-hydroxyethanesulfonic acid, formic acid, fumaric acid, galactaric acid, gentisic acid, glucoheptonic acid, gluconic acid, glucuronic acid, glutamic acid, glutaric acid, 2-oxo-glutaric acid, glycerophosphoric acid, glycolic acid, hippuric acid, isobutyric acid, lactic acid, lactobionic acid, lauric acid, maleic acid, malic acid, malonic acid, mandelic acid, methanesulfonic acid, mucic acid, naphthalene-1,5-disulfonic acid, naphthalene-2-sulfonic acid, 1-hydroxy-2-naphthoic acid, nicotinic acid, oleic acid, orotic acid, oxalic acid, palmitic acid, pamoic acid, propionic acid, pyroglutamic acid, pyruvic acid, salicylic acid, 4-aminosalicylic acid, sebacic acid, stearic acid, succinic acid, tartaric acid, thiocyanic acid, p-toluenesulfonic acid, trifluoroacetic acid, or undecylenic acid. Salts derived from inorganic bases may include, but are not limited to, sodium, potassium, lithium, ammonium, calcium, magnesium, iron, zinc, copper, manganese, and aluminum salts. Salts derived from organic bases may include, but are not limited to, salts of primary, secondary, or tertiary amines; substituted amines including naturally occurring substituted amines; cyclic amines; ammonia, isopropylamine, trimethylamine, diethylamine, triethylamine, tripropylamine, diethanolamine, ethanolamine, deanol, 2-dimethylaminoethanol, 2-diethylaminoethanol, dicyclohexylamine, lysine, arginine, histidine, caffeine, procaine, hydrabamine, choline, betaine, benethamine, benzathine, ethylenediamine, glucosamine, methylglucamine, theobromine, triethanolamine, tromethamine, purines, piperazine, piperidine, or N-ethylpiperidine.

In some embodiments, provided is a solvate of Compound 1, or a pharmaceutically acceptable salt, tautomer, isotope, or isomer thereof. In certain embodiments, the solvate is a hydrate. Thus, provided herein is a hydrate of Compound 1.

In some embodiments, provided is an isotope of Compound 1, or a pharmaceutically acceptable salt, solvate, tautomer, or isomer thereof. Thus, in some embodiments, provided herein is Compound 1 comprising one or more isotopically enriched atoms. Compound 1 may contain unnatural proportions of atomic isotopes at one or more of the atoms that constitute Compound 1. In some embodiments, the compound is isotopically-labeled, such as an isotopically-labeled Compound 1, or a pharmaceutically acceptable salt, solvate, tautomer, or isomer thereof, where a fraction of one or more atoms are replaced by an isotope of the same element. Exemplary isotopes that can be incorporated into Compound 1 or a pharmaceutically acceptable salt, solvate, tautomer, or isomer thereof include isotopes of hydrogen, carbon, nitrogen, oxygen, phosphorus, sulfur, and chlorine, such as ²H, ₃H, ¹¹C, ¹³C, ¹⁴C ¹³N, ¹⁵O, ¹⁷O, ³⁵S, ¹⁸F, and ³⁶Cl. Certain isotope labeled compounds (e.g. ³H and ¹⁴C) may be useful in compound or substrate tissue distribution study. Incorporation of heavier isotopes such as deuterium (²H) may, in some embodiments, afford certain therapeutic advantages resulting from greater metabolic stability, for example, increased in vivo half-life, or reduced dosage requirements.

Further provided herein is a pharmaceutical composition comprising Compound 1, or a pharmaceutically acceptable salt, solvate, tautomer, isotope, or isomer thereof, and a pharmaceutically acceptable excipient. A pharmaceutically acceptable excipient may include, for example, an adjuvant, carrier, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans. Pharmaceutically acceptable excipients may include, but are not limited to, water, NaCl, normal saline solutions, lactated Ringer's solution, normal sucrose, normal glucose, binders, fillers, disintegrants, lubricants, coatings, sweeteners, flavors, salt solutions (such as Ringer's solution), alcohols, oils, gelatins, carbohydrates (such as lactose, amylose or starch), fatty acid esters, hydroxymethycellulose, polyvinyl pyrrolidine, and colors.

As generally used herein, “pharmaceutically acceptable” refers to those compounds, materials, compositions, and/or dosage forms which are, within the scope of sound medical judgment, suitable for use in contact with the tissues, organs, and/or bodily fluids of human beings and animals without excessive toxicity, irritation, allergic response, or other problems or complications commensurate with a reasonable benefit/risk ratio.

### II. Methods of using Compound 1 and Pharmaceutical Compositions Comprising Compound 1

Provided herein are methods of using Compound 1, or a pharmaceutically acceptable salt, solvate, tautomer, isotope, or isomer thereof, or a pharmaceutical composition comprising any of the foregoing and a pharmaceutically acceptable excipient. These include methods of inhibiting a component of the SREBP pathway, such as an SREBP or SCAP; and methods of treating a disorder in a subject in need thereof. In some embodiments, the disorder is mediated by an SREBP or SCAP.

The terms “treat,” “treating,” or “treatment” refers to any indicia of success in the amelioration of an disorders, such as an injury, disease, pathology, or condition, including any objective or subjective parameter such as abatement; remission; diminishing of symptoms or making the injury, disease, disorder, pathology, or condition more tolerable to the subject; slowing or stopping the rate of degeneration, decline, or development; slowing the progression of the disorder (such as an injury, disease, pathology, or condition); making the final point of degeneration less debilitating; improving a subject's physical or mental well-being; or relieving or causing regression of the disorder (such as an injury, disease, pathology, or condition). The treatment of symptoms, including the amelioration of symptoms, can be based on objective or subjective parameters, which may include the results of a physical examination, a neuropsychiatric exam, and/or a psychiatric evaluation. Provided herein are methods of treating a hyperproliferative disorder. In some embodiments, the hyperproliferative disorder is cancer. Certain methods disclosed herein may treat cancer by, for example, decreasing the incidence of cancer, causing remission of cancer, slowing the rate of growth of cancer cells, slowing the rate of spread of cancer cells, reducing metastasis, or reducing the growth of metastatic tumors, reducing the size of one or more tumors, reducing the number of one or more tumors, or any combinations thereof.

The embodiments described herein for methods of treatment should also be considered to apply to the use of Compound 1, or a pharmaceutically acceptable salt, solvate, tautomer, isotope, or isomer thereof, for the treatment of disorders (such as an injury, disease, pathology, or condition); and the use of Compound 1, or a pharmaceutically acceptable salt, solvate, tautomer, isotope, or isomer thereof, for inhibiting an SREBP or inhibiting the proteolytic activation of an SREBP; and other uses of Compound 1, or a pharmaceutically acceptable salt, solvate, tautomer, isotope, or isomer thereof, as described herein; and the use of Compound 1, or a pharmaceutically acceptable salt, solvate, tautomer, isotope, or isomer thereof in the manufacture of medicaments.

### A. Inhibiting SREBP or SCAP

Provided herein are uses and methods of inhibiting a component of the SREBP pathway, such as an SREBP or SCAP. In some embodiments, a combination of an SREBP and SCAP is inhibited. Such methods may include contacting an SREBP with Compound 1, or a pharmaceutically acceptable salt, solvate, tautomer, isotope, or isomer thereof, or a pharmaceutical composition comprising any of the forgoing and a pharmaceutically acceptable excipient. Such methods may also include contacting SCAP with Compound 1, or a pharmaceutically acceptable salt, solvate, tautomer, isotope, or isomer thereof, or a pharmaceutical composition comprising any of the forgoing and a pharmaceutically acceptable excipient.

In certain embodiments, Compound 1, or a pharmaceutically acceptable salt, solvate, tautomer, isotope, or isomer thereof, is administered to a subject in need thereof to inhibit a component of the SREBP pathway. In other embodiments, a pharmaceutical composition comprising a pharmaceutically acceptable excipient and Compound 1, or a pharmaceutically acceptable salt, solvate, tautomer, isotope, or isomer thereof, is administered to the subject in need thereof. In certain embodiments, the amount of Compound 1 or a pharmaceutically acceptable salt, solvate, tautomer, isotope, or isomer thereof, relative to the subject's body mass, is between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof, is administered to a subject in need thereof to inhibit a component of the SREBP pathway. In certain embodiments, Compound 1 or a pharmaceutically acceptable salt, solvate, tautomer, isotope, or isomer thereof, is administered as a pharmaceutical composition, as described herein.

The component of the SREBP pathway that is inhibited by the methods and uses described herein may be an SREBP or SCAP. In some embodiments, an SREBP is inhibited. The SREBP may be, for example, an SREBP-1 (such as SREBP-1a or SREBP-1c) or SREBP-2. In certain variations, two or three of SREBP-1a, SREBP-1c, and SREBP-2 are inhibited. In some embodiments, the component is an SREBP-1. In other embodiments, the SREBP is SREBP-1a. In certain embodiments, the component is SREBP-1c. In still other embodiments, the SREBP is SREBP-2. In other embodiments, the component of the SREBP pathway is SCAP. In some embodiments, both an SREBP and SCAP are inhibited. In certain embodiments, two or three of SREBP-1a, SREBP-1c, and SREBP-2 are inhibited, and SCAP is inhibited.

Inhibition of a component of the SREBP pathway, such as an SREBP or SCAP, may include partial inhibition or full inhibition. Partial inhibition may include reducing the activity of a component of the SREBP pathway to a level that is still detectable. Full inhibition may include stopping all activity of a component of the SREBP pathway (such as stopping the activity of an SREBP or SCAP), or reducing the activity of a component of the SREBP pathway to a level below detection. Inhibition of a component of the SREBP pathway may be measured directly or indirectly, using any methods known in the art.

In some embodiments, inhibition of a component of the SREBP pathway is measured directly, for example by measuring the product of a reaction catalyzed by an SREBP pathway component. Inhibition of SREBP activation (for example, by inhibiting SCAP) may in some embodiments be demonstrated by western blotting and quantitatively assessing the levels of full-length and cleaved SREBP-1 and/or SREBP-2 proteins from a cell line (such as a hepatic cell lines) or primary cells (such as primary hepatocytes of mouse, rat or human origin).

In some embodiments, inhibition of a component of the SREBP pathway is measured indirectly, for example by measuring the level of expression of one or more genes that are regulated by SREBP. The inhibition of a component of the SREBP pathway, such as an SREBP or SCAP, may reduce the expression of one or more genes that are regulated by an SREBP, for example an SREBP-1 (such as SREBP-1a or SREBP-1c) or SREBP-2. SCAP plays a role in activating SREBPs, thus inhibiting the activity of SCAP may reduce the expression of one or more genes that are regulated by an SREBP. SREBP pathway inhibition may also be determined by assessing gene transcription levels of one or more target genes of SREBP-1 and/or SREBP-2, such as one or more of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, or ACACB. The transcription levels may be assessed, for example, by transcriptomic analysis, including but not limited to q-PCR. A reduction in one, two, three, four, five, or more of these genes may indicate inhibition of SREBP activation. This evaluation of endogenous SREBP gene expression may be assessed in cell lines (such as hepatic cell lines) or primary cells (such as primary hepatocytes of mouse, rat, or human origin). In some embodiments, the gene transcription levels of PCSK9 or PNPLA3, or a combination thereof, are evaluated.

Therefore, provided herein are uses and methods of reducing the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB, comprising contacting an SREBP or SCAP with Compound 1 or a pharmaceutically acceptable salt, solvate, tautomer, isotope, or isomer thereof. In some embodiments, the expression of PCSK9 is reduced. In other embodiments, the expression of PNPLA3 is reduced. In still further embodiments, both the expression of PCSK9 and PNPLA3 are reduced. In certain embodiments, one or more SREBP is contacted, for example an SREBP-1 (such as SREBP-1a or SREBP-1c) or SREBP-2, or any combinations thereof. In other embodiments, SCAP is contacted. In still further embodiments, one or more of SREBP-1a, SREBP-1c, SREBP-2, and SCAP is contacted. In certain embodiments, inhibition of a component of the SREBP pathway may treat a disorder mediated by an SREBP, such as the disorders as described herein. Thus, in certain embodiments, expression of one or more genes as described above is reduced in a subject in need thereof.

Another method of indirectly detecting SREBP pathway inhibition may include: Serum-starving a hepatic cell line (HepG2) expressing luciferase under the control of the LSS-promoter to induce SREBP activation and increased luciferase expression. The cells may then be treated with a compound, such as Compound 1, or a pharmaceutically acceptable salt, solvate, tautomer, isotope, or isomer thereof. Following treatment, a reduction of luciferase activity reflects inhibition of SREBP activation, and non-cytotoxicity of the compound can be assessed by LDH release.

### B. Treating a Disorder

Provided herein are uses and methods of treating a disorder in a subject in need thereof, comprising administering to the subject Compound 1, or a pharmaceutically acceptable salt, solvate, tautomer, isotope, or isomer thereof. In some embodiments, provided herein are uses and methods of treating a disorder in a subject in need thereof, comprising administering to the subject a pharmaceutical composition comprising Compound 1, or a pharmaceutically acceptable salt, solvate, tautomer, isotope, or isomer thereof, and a pharmaceutically acceptable excipient. In some embodiments, the disorder is mediated by an SREBP.

The uses and methods of treatment described herein may use Compound 1, or a pharmaceutically acceptable salt, solvate, tautomer, isotope, or isomer thereof; or a pharmaceutical composition comprising Compound 1, or a pharmaceutically acceptable salt, solvate, tautomer, isotope, or isomer thereof, and a pharmaceutically acceptable excipient.

### 1. Metabolic Disorders

In some embodiments, the disorder is a metabolic disorder, such as a disorder that affects lipid metabolism, cholesterol metabolism, or insulin metabolism. In certain embodiments, the disorder is related to lipid metabolism, cholesterol metabolism, or insulin metabolism, for example, liver disease as a result of the buildup of fat in the liver, or cardiovascular disease.

In some embodiments, the disorder is a liver disease, such as chronic liver disease. In some embodiments, the liver disease is mediated by a component of the SREBP pathway, such as an SREBP or SCAP. In some embodiments, the liver disease is mediated by an SREBP. In certain embodiments, the liver disease is mediated by a downstream gene target of an SREBP, such as PNPLA-3. In other embodiments, the liver disease is mediated by SCAP. Thus, in some embodiments, provided herein are uses and methods of treating a liver disease in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. The chronic liver disease may be, for example, primary alcoholic liver disease, nonalcoholic fatty liver disease (NAFLD), or nonalcoholic steatohepatitis (NASH). In some embodiments, the liver disease is liver fat, liver inflammation, or liver fibrosis, or a combination thereof.

In certain embodiments, the liver disease is non-alcoholic fatty liver disease (NAFLD). NAFLD is a group of conditions that are related to fat buildup in the liver. Non-alcoholic steatohepatitis (NASH) is a form of NAFLD which includes liver inflammation. In NASH, the liver inflammation may lead to liver damage and scarring, which can be irreversible, and it can also progress to cirrhosis and liver failure. NAFLD and NASH are associated with metabolic disorders such as obesity, dyslipidemia, insulin resistance, and type 2 diabetes. Other disorders associated with NAFLD and NASH include increased abdominal fat and high blood pressure. In some embodiments, NASH is mediated by a component of the SREBP pathway, such as an SREBP or SCAP.

In some embodiments, provided herein are uses and methods of treating NASH in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. Treatment of NASH may include reduction in average liver fat content, which may be evaluated, for example, by magnetic resonance imaging (MM), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation as evaluated through histological scoring of liver biopsy; reduction of liver fibrosis as evaluated through histological scoring of liver biopsy; reduction of liver fat content as evaluated through histological scoring of liver biopsy; or any combinations thereof. Treatment of NASH may be evaluated using the NAFLD activity score (NAS); or steatosis, activity, and fibrosis score (SAF); or other NASH diagnostic and/or scoring metrics (such as FIB4 or ELF).

Further provided herein are uses and methods of treating a disorder in a subject in need thereof, wherein the disorder is liver fibrosis associated with NASH, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. In some embodiments, the liver fibrosis is mediated by SREBP. Treatment of liver fibrosis may be evaluated, for example, by magnetic resonance imaging (MM), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation and/or fibrosis as evaluated through histological scoring of liver biopsy; or any combinations thereof.

Further provided herein are uses and methods of treating a disorder in a subject in need thereof, wherein the disorder is fatty liver disease, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. In some embodiments, the fatty liver disease is mediated by SREBP. In certain embodiments, a subject may have fatty liver disease when the fat content of the subject's liver is 5% or greater. In some embodiments, a subject with fatty liver disease has NASH, or liver fibrosis associated with NASH. In certain embodiments, a subject with fatty liver disease has not been diagnosed with NASH or liver fibrosis associated with NASH. Treatment of fatty liver disease may be evaluated, for example, by magnetic resonance imaging (MRI), magnetic resonance elastography (MRE), ultrasound, or computerized tomography (CT); reduction of the liver enzyme alanine aminotransferase (ALT); reduction of the liver enzyme aspartate aminotransferase (ALT); reduction of liver inflammation as evaluated through histological scoring of liver biopsy; reduction of liver fibrosis as evaluated through histological scoring of liver biopsy; reduction of liver fat content as evaluated through histological scoring of liver biopsy; or any combinations thereof.

In some embodiments of the uses and methods of treating liver disease provided herein, such as methods of treating liver fibrosis, fatty liver disease, or NASH, the subject is administered between about 0.01 mg/kg to about 100 mg/kg of Compound 1, or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof is administered to the subject in need thereof. In certain embodiments, Compound 1, or a pharmaceutically acceptable salt, solvate, tautomer, isotope, or isomer thereof, is administered as a pharmaceutical composition, as described herein.

Other metabolic disorders which may be treated with the compounds or pharmaceutical compositions described herein may include, for example, insulin resistance, hyperglycemia, diabetes mellitus, dyslipidemia, adiposopathy, obesity, and Metabolic Syndrome. In some embodiments, the metabolic disorder is mediated by a genetic factor. In other embodiments, the metabolic disorder is mediated by one or more environmental factors, such as a diet rich in fat, or a diet rich in sugar, or a combination thereof. In some embodiments, the metabolic disorder is mediated by SREBP. In some embodiments, the diabetes mellitus is type I diabetes. In certain embodiments, the diabetes mellitus is type II diabetes.

Provided herein are uses and methods of treating diabetes in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. Diabetes (also known as diabetes mellitus) refers to a disease or condition that is generally characterized by metabolic defects in production and utilization of glucose which result in the failure to maintain appropriate blood sugar levels in the body. In some embodiments, the diabetes is type II diabetes, which is characterized by insulin resistance, in which insulin loses its ability to exert its biological effects across a broad range of concentrations. In some embodiments, the diabetes is mediated by a component of the SREBP pathway, such as an SREBP or SCAP.

Further provided herein are uses and methods of treating insulin resistance in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. Insulin resistance has been hypothesized to unify the clustering of hypertension, glucose intolerance, hyperinsulinemia, increased levels of triglyceride, decreased HDL cholesterol, and central and overall obesity. “Metabolic Syndrome” refers to a similar clustering of conditions, which may include abdominal obesity, hypertension, high blood sugar, high serum triglycerides (such as elevated fasting serum triglycerides), and low HDL levels, and is associated with a risk of developing cardiovascular disease and/or type II diabetes. Further provided herein are uses and methods of treating Metabolic Syndrome in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. In some embodiments, the Metabolic Syndrome or insulin resistance is mediated by a component of the SREBP pathway, such as an SREBP or SCAP.

In some embodiments of the uses and methods of treating insulin resistance, hyperglycemia, diabetes mellitus, obesity, or Metabolic Syndrome provided herein, the subject is administered between about 0.01 mg/kg to about 100 mg/kg of Compound 1, or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof is administered to the subject in need thereof. In certain embodiments, Compound 1, or a pharmaceutically acceptable salt, solvate, tautomer, isotope, or isomer thereof, is administered as a pharmaceutical composition, as described herein.

In other embodiments, the metabolic disorder is dyslipidemia. Thus, in other embodiments, provided herein are uses and methods of treating dyslipidemia in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. Dyslipidemia refers to abnormal blood plasma levels of one or more lipids or one or more lipoproteins, or any combinations thereof. Dyslipidemia may include depressed levels or elevated levels of one or more lipids and/or one or more lipoproteins, or a combination of depressed and elevated levels (for example, elevated levels of one type of lipid and depressed levels of another type of lipid and/or lipoprotein). Dyslipidemia may include, but is not limited to, elevated low density lipoprotein cholesterol (LDL), elevated apolipoprotein B, elevated triglycerides (TGs), elevated lipoprotein(a), elevated apolipoprotein A, reduced high density lipoprotein cholesterol (HDL), or reduced apolipoprotein A1, or any combinations thereof. Dyslipidemia, such as abnormal cholesterol or abnormal TG levels, is associated with an increased risk for vascular disease (such as heart attack or stroke), atherosclerosis, and coronary artery disease. In some embodiments of the methods provided herein, the dyslipidemia is hyperlipidemia. Hyperlipidemia refers to the presence of an abnormally elevated level of lipids in the blood, and may include (1) hypercholesterolemia (an elevated cholesterol level); (2) hypertriglyceridemia, (an elevated triglyceride level); and (3) combined hyperlipidemia, (a combination of hypercholesterolemia and hypertriglyceridemia). Dyslipidemia may arise from a combination of genetic predisposition and diet, and may be associated with being overweight, diabetes, or Metabolic Syndrome. Lipid disorders may also arise as the result of certain medications (such as those used for anti-rejection regimens in people who have had organ or tissue transplants). In some embodiments, the dyslipidemia, such as hyperlipidemia, is mediated by a component of the SREBP pathway, such as an SREBP or SCAP. Thus, in some embodiments, provided herein are uses and methods of reducing cholesterol levels, modulating cholesterol metabolism, modulating cholesterol catabolism, modulating the absorption of dietary cholesterol, reversing cholesterol transport, or lowering triglycerides in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient.

In some embodiments of the uses and methods of treating dyslipidemia provided herein, such as reducing cholesterol levels, modulating cholesterol metabolism, modulating cholesterol catabolism, modulating the absorption of dietary cholesterol, reversing cholesterol transport, or lowering triglycerides in a subject in need thereof as provided herein, the subject is administered between about 0.01 mg/kg to about 100 mg/kg of Compound 1, or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof is administered to the subject in need thereof. In certain embodiments, Compound 1 or a pharmaceutically acceptable salt, solvate, tautomer, isotope, or isomer thereof, is administered as a pharmaceutical composition, as described herein.

In still other embodiments, provided herein of treating adiposopathy in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. In some embodiments, the adiposopathy is associated with Metabolic Syndrome. In some embodiments, the adiposopathy is mediated by a component of the SREBP pathway, such as an SREBP or SCAP.

In certain embodiments, provided herein is are uses and methods of treating gallstones in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient. Gallstones may be associated with gallbladder inflammation, pancreas inflammation, or liver inflammation. In certain embodiments, the gallstones are cholesterol gallstones, which may form when bile contains a high concentration of cholesterol and not enough bile salts. In some embodiments, the gallstones, which may include cholesterol gallstone disease, is mediated by a component of the SREBP pathway, such as an SREBP or SCAP.

In other embodiments, the disorder is pancreatitis. In yet other embodiments, the disorder is endotoxic shock, systemic inflammation, or xanthoma. In still further embodiments, the disorder is atherosclerosis, coronary artery disease, angina pectoris, carotid artery disease, stroke, or cerebral arteriosclerosis. In certain embodiments, any of the foregoing disorders are mediated by a component of the SREBP pathway, such as an SREBP or SCAP.

In some embodiments of the uses and methods of treating gall stones, pancreatitis, endotoxic shock, systemic inflammation, xanthoma, atherosclerosis, coronary artery disease, angina pectoris, carotid artery disease, stroke, or cerebral arteriosclerosis provided herein, the subject is administered between about 0.01 mg/kg to about 100 mg/kg of Compound 1, or a pharmaceutically acceptable salt, solvate, tautomer, isotope, or isomer thereof, relative to the body mass of the subject. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof is administered to the subject in need thereof. In certain embodiments, Compound 1, or pharmaceutically acceptable salt, solvate, tautomer, isotope, or isomer thereof, is administered as a pharmaceutical composition, as described herein.

In some embodiments of any of the above embodiments, the subject is overweight, obese, has insulin resistance, is pre-diabetic or has type II diabetes. In certain embodiments of any of the preceding embodiments, the subject has NASH.

### 2. Hyperproliferative Disorders

In other embodiments, the disorder is a hyperproliferative disorder. Thus, in some embodiments, provided herein are uses and methods of treating a hyperproliferative disorder in a subject in need thereof, comprising administering to the subject Compound 1, or a pharmaceutically acceptable salt, solvate, tautomer, isotope, or isomer thereof; or a pharmaceutical composition comprising any of the foregoing and a pharmaceutically acceptable excipient.

As described above, the metabolism of fatty acids, cholesterol, and triglycerides may play a role in hyperproliferative disorders, such as cancer. Often, during transformation of non-cancerous cells to cancerous cell, cell metabolism shifts from catabolic to anabolic processes. Depending on the type of tumor, the tumor cells may synthesize up to 95% of the saturated and mono-unsaturated fatty acids. Some cancers exhibit increased synthesis of fatty acids and other lipids (such as cholesterol), and steroids (such as androgens). Elevated fatty acid synthase (FAS) expression may induce progression to S phase in cancer cells, and inhibition of FAS expression may reduce cell growth and may induce apoptosis. Thus, components of the SREBP pathway may play a role in hyperproliferative disorders.

Hyperproliferative disorders, which are disorders associated with some degree of abnormal cell proliferation, may be benign or malignant. Benign hyperproliferative disorders may include pre-cancerous disorders.

In some embodiments of the uses and methods provided herein, the disorder is a benign hyperproliferative disorder. In some embodiments, the benign hyperproliferative disorder is mediated by a component of the SREBP pathway, such as an SREBP or SCAP. In other embodiments, the disorder is a malignant hyperproliferative disorder. In some embodiments, the malignant hyperproliferative disorder is mediated by a component of the SREBP pathway, such as an SREBP or SCAP.

In some embodiments, the hyperproliferative disorder is breast cancer, liver cancer, ovarian cancer, pancreatic cancer, or prostate cancer.

In some embodiments, the hyperproliferative disorder is a soft tissue sarcoma, bladder cancer, endometrial cancer, skin cancer, colon cancer, hematologic cancer, placenta cancer, brain cancer, kidney cancer, lung cancer, or bone cancer. Sarcoma can include cancers that begin in the bones and in the soft tissues. Sarcoma includes, for example, connective tissue cancers, such as muscle cancers.

In some embodiments of the uses and methods of treating a hyperproliferative disorder in a subject in need thereof, as described herein, between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof, relative to the body mass of the subject, is administered to the subject in need thereof. In certain embodiments, Compound 1, or pharmaceutically acceptable salt, solvate, tautomer, isotope, or isomer thereof, is administered as a pharmaceutical composition, as described herein.

### III. Dosing and Methods of Administration

The dose of Compound 1, or a pharmaceutically acceptable salt, solvate, tautomer, isotope, or isomer thereof, administered to a subject in need thereof according to any of the disclosed uses and methods may vary between Compound 1 and the particular pharmaceutically acceptable salt, solvate, tautomer, isotope, or isomer thereof; the method of administration; the particular disorder being treated; and the characteristics of the subject (such as weight, sex, and/or age). In some embodiments, the amount of Compound 1, or a pharmaceutically acceptable salt, solvate, tautomer, isotope, or isomer thereof is a therapeutically effective amount.

The effective amount of Compound 1, or pharmaceutically acceptable salt, solvate, tautomer, isotope, or isomer thereof, relative to the subject's body mass, may in some embodiments be between about 0.01 mg/kg to about 100 mg/kg. In some embodiments, about 0.7 mg to about 7 g daily, or about 7 mg to about 350 mg daily, or about 350 mg to about 1.75 g daily, or about 1.75 to about 7 g daily of Compound 1, or a pharmaceutically acceptable salt, solvate, tautomer, isotope, or isomer thereof is administered to a subject in need thereof. In certain embodiments, Compound 1, or a pharmaceutically acceptable salt, solvate, tautomer, isotope, or isomer thereof, is administered as a pharmaceutical composition, as described herein.

Any of the uses and methods provided herein may comprise administering to a subject in need therein a pharmaceutical composition that comprises an effective amount of Compound 1, or a corresponding amount of a pharmaceutically acceptable salt, solvate, tautomer, isotope, or isomer thereof, and a pharmaceutically acceptable excipient.

Compound 1, or a pharmaceutically acceptable salt, solvate, tautomer, isotope, or isomer thereof as provided herein, or a pharmaceutical composition comprising any of these and a pharmaceutically acceptable excipient as provided herein, may be administered to a subject via any suitable route, including, for example, intravenous, intramuscular, subcutaneous, oral, or transdermal routes.

In certain embodiments, the provided herein are uses and methods of treating a disorder in subject in need thereof by parenterally administering to the subject an effective amount of Compound 1, or a pharmaceutically acceptable salt, solvate, tautomer, isotope, or isomer thereof as provided herein, or a pharmaceutical composition comprising an effective amount of any of the foregoing and a pharmaceutically acceptable excipient as provided herein. In some embodiments, the disorder is a hyperproliferative disorder. In certain embodiments, the hyperproliferative disorder is cancer. In other embodiments, the disorder is fatty liver disease. In certain embodiments, the disorder is NASH. In some embodiments, the route of administration is intravenous, intra-arterial, intramuscular, or subcutaneous. In some embodiments, the route of administration is transdermal.

In certain embodiments, provided herein are pharmaceutical compositions comprising Compound 1, or a pharmaceutically acceptable salt, solvate, tautomer, isotope, or isomer thereof, and a pharmaceutically acceptable excipient, for the use in treating a disorder as described herein. In some embodiments, the disorder is prevented, or the onset delayed, or the development delayed. In some embodiments, the disorder is a hyperproliferative disorder. In certain embodiments, the hyperproliferative disorder is cancer. In some embodiments, the disorder is fatty liver disease. In certain embodiments, the disorder is NASH. In certain embodiments, the composition comprises a pharmaceutical formulation which is present in a one or more unit dosage forms, for example one, two, three, four, or more unit dosage forms.

### IV. Kits

Also provided are articles of manufacture comprising Compound 1, or a pharmaceutically acceptable salt, solvate, tautomer, isotope, or isomer thereof, or pharmaceutical compositions comprising same, or unit dosages comprising any of these, as described herein in suitable packaging for use in the methods described herein. Suitable packaging may include, for example, vials, vessels, ampules, bottles, jars, flexible packaging, and the like. An article of manufacture may further be sterilized and/or sealed kits.

Further provided herein are kits comprising Compound 1, or a pharmaceutically acceptable salt, solvate, tautomer, isotope, or isomer thereof, or a pharmaceutical composition comprising same and a pharmaceutically acceptable excipient. The kits may be used in any of the methods described herein. In some embodiments, the kit further comprises instructions. The kits may be used for any one or more of the uses described herein, and, accordingly, may contain instructions for the treatment of a hyperproliferative disease (such as cancer), fatty liver disease, or NASH. The kits may comprise one or more containers. Each component (if there is more than one component) may be packaged in separate containers or some components may be combined in one container where cross-reactivity and shelf life permit.

The kits may be in unit dosage forms, bulk packages (e.g., multi-dose packages) or subunit doses. For example, kits may be provided that contain sufficient dosages of Compound 1, or a pharmaceutically acceptable salt, solvate, tautomer, isotope, or isomer thereof, as disclosed herein and/or a second pharmaceutically active compound useful for a disorder detailed herein to provide effective treatment of a subject for an extended period, such as one week, 2 weeks, 3 weeks, 4 weeks, 6 weeks, 8 weeks, 3 months, 4 months, 5 months, 7 months, 8 months, 9 months, or more. Kits may also include multiple unit doses of Compound 1, or a pharmaceutically acceptable salt, solvate, tautomer, isotope, or isomer thereof, and instructions for use, and be packaged in quantities sufficient for storage and use in pharmacies (e.g., hospital pharmacies or compounding pharmacies).

The kits may optionally include a set of instructions, generally written instructions, although electronic storage media (e.g., magnetic diskette or optical disk) containing instructions are also acceptable, relating to the use of component(s) of the methods as described herein. The instructions included with the kit may include information as to the components and their administration to an individual.

The present description sets forth numerous exemplary configurations, methods, parameters, and the like. It should be recognized, however, that such description is not intended as a limitation on the scope of the present disclosure, but is instead provided as a description of exemplary embodiments.

### ENUMERATED EMBODIMENTS

Embodiment I-1. The compound (3-chloro-4-(4-(2-(2-hydroxypropan-2-yl)pyridin-4-yl)thiophen-2-yl)phenyl)(4-hydroxypiperidin-1- yl)methanone:

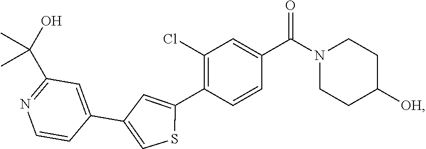 [SMILES: CC(C)(O)c1cc(-c2csc(-c3ccc(C(=O)N4CCC(O)CC4)cc3Cl)c2)ccn1]

or a pharmaceutically acceptable salt, solvate, tautomer, isotope, or isomer thereof.

Embodiment I-2. A pharmaceutical composition, comprising the compound Embodiment I-1, or a pharmaceutically acceptable salt, solvate, tautomer, isotope, or isomer thereof, and a pharmaceutically acceptable excipient.

Embodiment I-3. A method of inhibiting a sterol regulatory element-binding protein (SREBP), comprising contacting the SREBP or contacting an SREBP cleavage activating-protein (SCAP) with the compound of Embodiment I-1, or a pharmaceutically acceptable salt, solvate, tautomer, isotope, or isomer thereof, or the pharmaceutical composition of Embodiment I-2.

Embodiment I-4. A method of inhibiting the proteolytic activation of a sterol regulatory element-binding protein (SREBP), comprising contacting an SREBP cleavage activating-protein (SCAP) with the compound of Embodiment I-1, or a pharmaceutically acceptable salt, solvate, tautomer, isotope, or isomer thereof, or the pharmaceutical composition of Embodiment I-2.

Embodiment I-5. A method of treating a disorder in a subject in need thereof, wherein the disorder is mediated by a sterol regulatory element-binding protein (SREBP), comprising administering to the subject an effective amount of the compound of Embodiment I-1, or a pharmaceutically acceptable salt, solvate, tautomer, isotope, or isomer thereof, or the pharmaceutical composition of Embodiment I-2.

Embodiment I-6. A method of treating a disorder in a subject in need thereof, comprising administering to the subject an effective amount of the compound of Embodiment I-1, or a pharmaceutically acceptable salt, solvate, tautomer, isotope, or isomer thereof, or the pharmaceutical composition of Embodiment I-2.

Embodiment I-7. The method of any one of Embodiments I-3 to 1-5, wherein the SREBP is an SREBP-1.

Embodiment I-8. The method of Embodiment I-7, wherein the SREBP-1 is SREBP-1a.

Embodiment I-9. The method of Embodiment I-7, wherein the SREBP-1 is SREBP-1c.

Embodiment I-10. The method of any one of Embodiments I-3 to 1-5, wherein the SREBP is SREBP-2.

Embodiment I-11. The method of any one of Embodiments I-3 to I-10, wherein SREBP is inhibited in a subject in need thereof.

Embodiment I-12. The method of any one of Embodiments I-3 to I-11, wherein SCAP is inhibited in a subject in need thereof.

Embodiment I-13. The method of any one of Embodiments I-3 to I-12, comprising contacting SREBP or SCAP with the compound, or pharmaceutically acceptable salt, solvate, tautomer, isotope, or isomer thereof, or the pharmaceutical composition, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof, or the pharmaceutical composition.

Embodiment I-14. The method of Embodiment I-5 or I-6, wherein the disorder is Metabolic Syndrome, type 2 diabetes, obesity, liver disease, insulin resistance, adiposopathy, or dyslipidemia.

Embodiment I-15. The method of Embodiment I-14, wherein the dyslipidemia is hypertriglyceridemia or elevated cholesterol levels.

Embodiment I-16. The method of Embodiment I-14, wherein the liver disease is nonalcoholic steatohepatitis, liver fibrosis, or liver inflammation, or a combination thereof.

Embodiment I-17. The method of Embodiment I-5 or I-6, wherein the disorder is a hyperproliferative disorder.

Embodiment I-18. The method of Embodiment I-17, wherein the hyperproliferative disorder is cancer.

Embodiment I-19. The method of Embodiment I-18, wherein the cancer is breast cancer, liver cancer, ovarian cancer, pancreatic cancer, or prostate cancer.

Embodiment I-19-A. The method of Embodiment I-18, wherein the cancer is breast cancer, liver cancer, ovarian cancer, pancreatic cancer, prostate cancer, a soft tissue sarcoma, bladder cancer, endometrial cancer, skin cancer, colon cancer, hematologic cancer, placenta cancer, brain cancer, kidney cancer, lung cancer, or bone cancer.

Embodiment I-20. The method of Embodiment I-5 or I-6, wherein the disorder is endotoxic shock, systemic inflammation, or atherosclerosis.

Embodiment I-21. Use of the compound of Embodiment I-1, or a pharmaceutically acceptable salt, solvate, tautomer, isotope, or isomer thereof, in the manufacture of a medicament for inhibiting a sterol regulatory element-binding protein (SREBP) in a subject in need thereof.

Embodiment I-22. The use of Embodiment I-21, wherein the inhibiting comprises contacting the SREBP or contacting an SREBP cleavage activating-protein (SCAP) with the compound or pharmaceutically acceptable salt, solvate, tautomer, isotope, or isomer thereof.

Embodiment I-23. Use of the compound of Embodiment I-1, or a pharmaceutically acceptable salt, solvate, tautomer, isotope, or isomer thereof, in the manufacture of a medicament for inhibiting the proteolytic activation of a sterol regulatory element-binding protein (SREBP) in a subject in need thereof.

Embodiment I-24. The use of Embodiment I-23, wherein the inhibiting comprises contacting an SREBP cleavage activating-protein (SCAP) with the compound or pharmaceutically acceptable salt, solvate, tautomer, isotope, or isomer thereof.

Embodiment I-25. Use of the compound of Embodiment I-1, or a pharmaceutically acceptable salt, solvate, tautomer, isotope, or isomer thereof, in the manufacture of a medicament for treating a disorder in a subject in need thereof, wherein the disorder is mediated by a sterol regulatory element-binding protein (SREBP).

Embodiment I-26. Use of the compound of Embodiment I-1, or a pharmaceutically acceptable salt, solvate, tautomer, isotope, or isomer thereof, in the manufacture of a medicament for treating a disorder in a subject in need thereof.

Embodiment I-27. The use of any one of Embodiments I-21 to I-25, wherein the SREBP is an SREBP-1.

Embodiment I-28. The use of Embodiment I-27, wherein the SREBP-1 is SREBP-1a.

Embodiment I-29. The use of Embodiment I-27, wherein the SREBP-1 is SREBP-1c.

Embodiment I-30. The use of any one of Embodiments I-21 to I-25, wherein the SREBP is SREBP-2.

Embodiment I-31. The use of any one of Embodiments I-21 to I-30, wherein SREBP is inhibited in a subject in need thereof.

Embodiment I-32. The use of any one of Embodiments I-21 to I-31, wherein SCAP is inhibited in a subject in need thereof.

Embodiment I-33. The use of any one of Embodiments I-21 to I-32, wherein an SREBP or SCAP is contacted with the compound, or pharmaceutically acceptable salt, solvate, tautomer, isotope, or isomer thereof, and the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SCSDL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

Embodiment I-34. The use of Embodiment I-25 or I-26, wherein the disorder is Metabolic Syndrome, type 2 diabetes, obesity, liver disease, insulin resistance, adiposopathy, or dyslipidemia.

Embodiment I-35. The use of Embodiment I-34, wherein the dyslipidemia is hypertriglyceridemia or elevated cholesterol levels.

Embodiment I-36. The use of Embodiment I-34, wherein the liver disease is nonalcoholic steatohepatitis, liver fibrosis, or liver inflammation, or a combination thereof.

Embodiment I-37. The use of Embodiment I-25 or I-26, wherein the disorder is a hyperproliferative disorder.

Embodiment I-38. The use of Embodiment I-37, wherein the hyperproliferative disorder is cancer.

Embodiment I-39. The use of Embodiment I-38, wherein the cancer is breast cancer, liver cancer, ovarian cancer, pancreatic cancer, or prostate cancer.

Embodiment I-39-A. The use of Embodiment I-38, wherein the cancer is breast cancer, liver cancer, ovarian cancer, pancreatic cancer, prostate cancer, a soft tissue sarcoma, bladder cancer, endometrial cancer, skin cancer, colon cancer, hematologic cancer, placenta cancer, brain cancer, kidney cancer, lung cancer, or bone cancer.

Embodiment I-40. The use of Embodiment I-25 or I-26, wherein the disorder is endotoxic shock, systemic inflammation, or atherosclerosis.

Embodiment I-41. Use of the compound of Embodiment I-1, or a pharmaceutically acceptable salt, solvate, tautomer, isotope, or isomer thereof, or the pharmaceutical composition of Embodiment I-2, for inhibiting a sterol regulatory element-binding protein (SREBP).

Embodiment I-42. The use of Embodiment I-41, wherein the inhibiting comprises contacting the SREBP or contacting an SREBP cleavage activating-protein (SCAP) with the compound or pharmaceutically acceptable salt, solvate, tautomer, isotope, or isomer thereof.

Embodiment I-43. Use of the compound of Embodiment I-1, or a pharmaceutically acceptable salt, solvate, tautomer, isotope, or isomer thereof, or the pharmaceutical composition of Embodiment I-2, for inhibiting the proteolytic activation of a sterol regulatory element-binding protein (SREBP).

Embodiment I-44. The use of Embodiment I-43, wherein the inhibiting comprises contacting an SREBP cleavage activating-protein (SCAP) with the compound or pharmaceutically acceptable salt, solvate, tautomer, isotope, or isomer thereof.

Embodiment I-45. Use of the compound of Embodiment I-1, or a pharmaceutically acceptable salt, solvate, tautomer, isotope, or isomer thereof, or the pharmaceutical composition of Embodiment I-2, for treating a disorder in a subject in need thereof, wherein the disorder is mediated by a sterol regulatory element-binding protein (SREBP).

Embodiment I-46. Use of the compound of Embodiment I-1, or a pharmaceutically acceptable salt, solvate, tautomer, isotope, or isomer thereof, or the pharmaceutical composition of Embodiment I-2, for treating a disorder in a subject in need thereof.

Embodiment I-47. The use of any one of Embodiments I-41 to I-45, wherein the SREBP is an SREBP-1.

Embodiment I-48. The use of Embodiment I-47, wherein the SREBP-1 is SREBP-1a.

Embodiment I-49. The use of Embodiment I-47, wherein the SREBP-1 is SREBP-1c.

Embodiment I-50. The use of any one of Embodiments I-41 to I-45, wherein the SREBP is SREBP-2.

Embodiment I-51. The use of any one of Embodiments I-41 to I-50, wherein SREBP is inhibited in a subject in need thereof.

Embodiment I-52. The use of any one of Embodiments I-41 to I-51, wherein SCAP is inhibited in a subject in need thereof.

Embodiment I-53. The use of any one of Embodiments I-41 to I-52, wherein an SREBP or SCAP is contacted with the compound, or pharmaceutically acceptable salt, solvate, tautomer, isotope, or isomer thereof, or the pharmaceutical composition, and the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SCSDL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof, or the pharmaceutical composition.

Embodiment I-54. The use of Embodiment I-45 or I-46, wherein the disorder is Metabolic Syndrome, type 2 diabetes, obesity, liver disease, insulin resistance, adiposopathy, or dyslipidemia.

Embodiment I-55. The use of Embodiment I-54, wherein the dyslipidemia is hypertriglyceridemia or elevated cholesterol levels.

Embodiment I-56. The use of Embodiment I-55, wherein the liver disease is nonalcoholic steatohepatitis, liver fibrosis, or liver inflammation, or a combination thereof.

Embodiment I-57. The use of Embodiment I-45 or I-46, wherein the disorder is a hyperproliferative disorder.

Embodiment I-58. The use of Embodiment I-57, wherein the hyperproliferative disorder is cancer.

Embodiment I-59. The use of Embodiment I-58, wherein the cancer is breast cancer, liver cancer, ovarian cancer, pancreatic cancer, or prostate cancer.

Embodiment I-59-A. The use of Embodiment I-58, wherein the cancer is breast cancer, liver cancer, ovarian cancer, pancreatic cancer, prostate cancer, a soft tissue sarcoma, bladder cancer, endometrial cancer, skin cancer, colon cancer, hematologic cancer, placenta cancer, brain cancer, kidney cancer, lung cancer, or bone cancer.

Embodiment I-60. The use of Embodiment I-45 or I-46, wherein the disorder is endotoxic shock, systemic inflammation, or atherosclerosis.

Embodiment I-61. A method of treating non-alcoholic steatohepatitis (NASH) in a subject in need thereof, comprising administering to the subject an effective amount of the compound of Embodiment I-1, or a pharmaceutically acceptable salt, solvate, tautomer, isotope, or isomer thereof, or the pharmaceutical composition of Embodiment I-2.

Embodiment I-62. Use of the compound of Embodiment I-1, or a pharmaceutically acceptable salt, solvate, tautomer, isotope, or isomer thereof, or the pharmaceutical composition of Embodiment I-2, for treating non-alcoholic steatohepatitis (NASH) in a subject in need thereof.

Embodiment I-63. Use of the compound of Embodiment I-1, or a pharmaceutically acceptable salt, solvate, tautomer, isotope, or isomer thereof, or the pharmaceutical composition of Embodiment I-2, in the manufacture of a medicament for treating non-alcoholic steatohepatitis (NASH) in a subject in need thereof.

Embodiment I-64. A method of treating a hyperproliferative disorder in a subject in need thereof, comprising administering to the subject an effective amount of the compound of Embodiment I-1, or a pharmaceutically acceptable salt, solvate, tautomer, isotope, or isomer thereof, or the pharmaceutical composition of Embodiment I-2.

Embodiment I-65. Use of the compound of Embodiment I-1, or a pharmaceutically acceptable salt, solvate, tautomer, isotope, or isomer thereof, or the pharmaceutical composition of Embodiment I-2, for treating a hyperproliferative disorder in a subject in need thereof.

Embodiment I-66. Use of the compound of Embodiment I-1, or a pharmaceutically acceptable salt, solvate, tautomer, isotope, or isomer thereof, or the pharmaceutical composition of Embodiment I-2, in the manufacture of a medicament for treating a hyperproliferative disorder in a subject in need thereof.

Embodiment I-67. The compound of Embodiment I-1, or a pharmaceutically acceptable salt, solvate, tautomer, isotope, or isomer thereof, for use in inhibiting a sterol regulatory element-binding protein (SREBP).

Embodiment I-68. The compound of Embodiment I-1, or a pharmaceutically acceptable salt, solvate, tautomer, isotope, or isomer thereof, for use in inhibiting the proteolytic activation of a sterol regulatory element-binding protein (SREBP).

Embodiment I-69. The compound of Embodiment I-1, or a pharmaceutically acceptable salt, solvate, tautomer, isotope, or isomer thereof, for use in treating a disorder in a subject in need thereof, wherein the disorder is mediated by a sterol regulatory element binding protein (SREBP).

Embodiment I-70. The compound of Embodiment I-1, or a pharmaceutically acceptable salt, solvate, tautomer, isotope, or isomer thereof, for use in treating a disorder in a subject in need thereof.

Embodiment I-71. The compound of Embodiment I-1, or a pharmaceutically acceptable salt, solvate, tautomer, isotope, or isomer thereof, for use in treating non-alcoholic steatohepatitis (NASH) in a subject in need thereof.

Embodiment I-72. The compound of Embodiment I-1, or a pharmaceutically acceptable salt, solvate, tautomer, isotope, or isomer thereof, for use in treating a hyperproliferative disorder in a subject in need thereof.

### EXAMPLES

The following Examples are merely illustrative and are not meant to limit any embodiments of the present disclosure in any way.

In some instances, Biological Examples 1-6 compare Compound 1 to its one-carbon homolog, Compound 2, in order to show that small changes in compound structure might lead to unexpected changes to in vitro and in vivo biological activity.

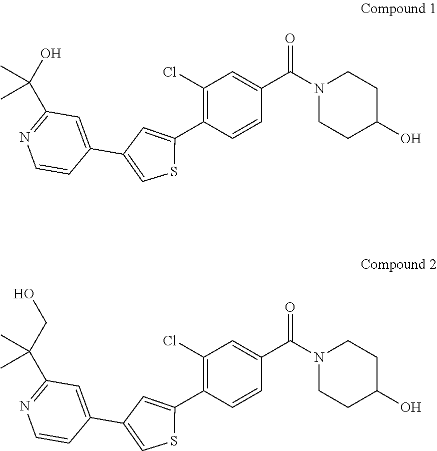 [SMILES: CC(C)(O)c1cc(-c2csc(-c3ccc(C(=O)N4CCC(O)CC4)cc3Cl)c2)ccn1 ; CC(C)(CO)c1cc(-c2csc(-c3ccc(C(=O)N4CCC(O)CC4)cc3Cl)c2)ccn1]

### Synthesis Example 1: Synthesis of Compound 1

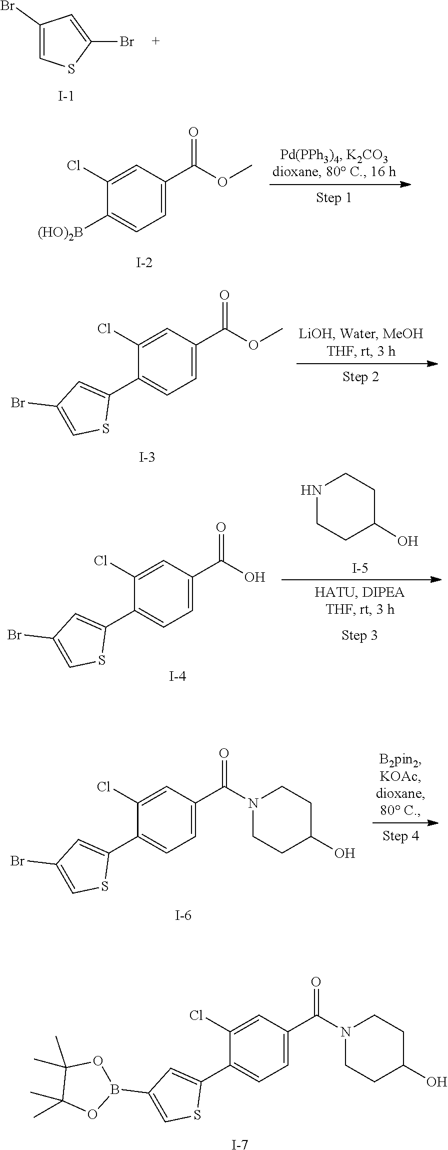

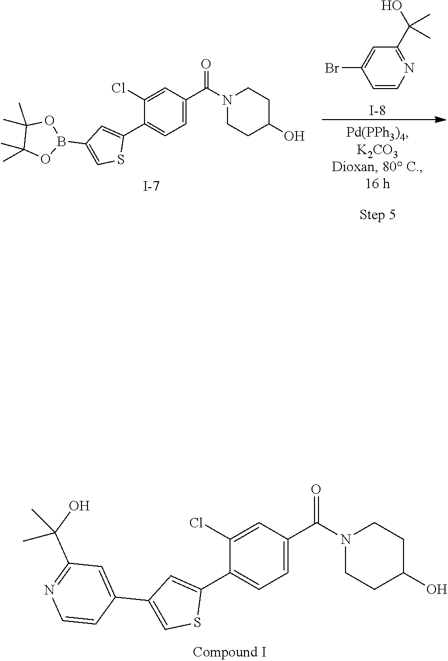

Compound 1 was synthesized according to the above scheme.

Step 1—Synthesis of Compound 1-3: A stirred solution of compound I-1 (500 mg, 2.083 mmol), compound 1-2 (401.00 mg, 1.88 mmol), and potassium carbonate (877.30 mg, 6.249 mmol) in water (1.5 mL) and 1,4-dioxane (5.0 mL), in 48 ml glass seal tube, was purged with nitrogen gas for 20 minutes. After adding palladium tetrakis (240.307 mg, 0.208 mmol) the reaction mixture was purged with nitrogen gas for 20 minutes, and the reaction mass was heated to 80° C. for 16 h. The reaction progress was monitored by TLC (TLC silica gel plate, UV to visualize the spot) and LCMS. After completion of the reaction, the mixture was cooled 25° C.-30° C. and filtered through a bed of celite and washed with ethyl acetate (30 mL). The combined organic layers were concentrated under reduced pressure to afford crude (800 mg). The crude compound (800 mg) was purified through 230-400 silica gel (column chromatography) using 15-20% ethyl acetate in petroleum ether as eluent. The appropriate fractions were collected and concentrated under reduced pressure to afford the product I-3 (440 mg, 65% yield) as an off-white solid. TLC system: 30% ethyl acetate in pet ether (R_{f} value=0.50). ¹H NMR (400 MHz, CDCl3): δ8.17 (d, J=2.00 Hz, 1H), 7.96 (dd, J=1.60, 8.00 Hz, 1H), 7.60 (d, J=8.00 Hz, 1H), 7.42 (d, J=1.60 Hz, 1H), 7.38 (d, J=1.60 Hz, 1H), 3.97 (s, 3H).

Step 2—Synthesis of Compound 1-4: To a stirred solution of compound 1-3 (440 mg, 1.337 mmol) in THF: MeOH: H₂O was added LiOH.hydrate (140.5 mg, 3.343 mmol) at 0-5° C. and the reaction mass was stirred for 2 h at RT. The reaction progress was monitored by TLC (TLC silica gel plate, UV to visualize the spot) and LCMS. After completion of reaction, solvent from the reaction mixture was evaporated completely, water was added, and the mixture was acidified with 1N HCl and extracted with ethyl acetate (30 mL). The combined organic layers were concentrated under reduced pressure to afford the product I-4 (380 mg, 65% yield) as an off-white solid. TLC system: 10% MeOH in MDC (R_{f} value=0.20). ¹H NMR (400 MHz, CDCl3): δ13.49 (s, 1H), 8.04 (s, 1H), 7.93-7.92 (m, 2H), 7.82 (d, J=8.0 Hz, 1H), 7.61 (s, 1H); LCMS: 89.66% (m/z=316.8 [M+H]).

Step 3—Synthesis of Compound I-6: To a stirred solution of compound I-4 (360 mg, 1.139 mmol) in THF was added HATU (657.8 mg, 1.708 mmol) at 0-5° C. followed by DIPEA (440.8 mg, 3.417 mmol). Then the reaction mixture was stirred at 0-5° C. for 30 mins followed by addition of piperidin-4-ol (172.6 mg, 1.708 mmol). The reaction mixture was stirred at RT for 16 h, and progress of the reaction was monitored by TLC (TLC silica gel plate, UV to visualize the spot) and LCMS. After completion. the reaction mass was quenched with with ice water and extracted with ethyl acetate (100 mL). The organic layer was washed with water and brine, and was concentrated under reduced pressure to afford crude material (600 mg). The crude compound (600 mg) was purified through 230-400 silica gel (column chromatography) using 55-60% ethyl acetate in petroleum ether as eluent. The appropriate fractions were collected and concentrated under reduced pressure to afford the product I-6 (390 mg, 56% yield) as an off-white solid. TLC system: 70% EtOAc in petroelum ether (R_{f} value=0.3). ¹H NMR (400 MHz, DMSO-d6): δ7.54-7.52 (m, 2H), 7.33-7.26 (m, 3H), 5.94 (s, 1H), 5.30 (br s, 1H), 4.00-4.01 (m, 1H), 3.69-3.66 (m, 2H), 3.29 (m, 2H), 2.05-1.85 (m, 2H), 1.60-1.50 (m, 2H). LCMS: 83.24% (m/z=400.17[M+H]).

Step 4—Synthesis of Compound I-7: A stirred solution of compound I-6 (390 mg, 0.9789 mmol), bispinacolato diboron (371.87 mg, 1.469 mmol), and potassium acetate (289.14 mg, 2.9367) in 1,4-dioxane (8 mL), in 48 ml sealed glass tube, was purged with nitrogen gas for 10 min. After adding PdCl₂(dppf) DCM adduct (79.87 mg, 0.0979 mmol), the reaction mixture was purged with nitrogen gas for 20 minutes, and the reaction mass was heated at 80° C. for 16 h. The reaction progress was monitored by TLC (TLC silica gel plate, UV to visualize the spot) and LCMS. After completion of the reaction, the mixture was cooled 25° C.-30° C. and filtered through a bed of celite and washed with ethyl acetate (2×30 mL). The combined organic layers were directly concentrated under reduced pressure to afford crude (800 mg). The crude compound (800 mg) was co-distilled with methanol thrice (each with 10 ml), and the material was stirred with 5% ethyl acetate in pet ether, then decanted and evaporated completely to get the desired boronate (320 mg, 42% yield). This was used in the next step without further purification. TLC system: 10% MeOH in DCM (R_{f} value=0.3). LCMS: 80.54% (m/z=448.1 [M+H]) and (m/z=366.1 [M+H], mass of boronic acid).

Step 5—Synthesis of Compound 1: A stirred solution of compound I-7 (300 mg, 0.6696 mmol), compound I-8 (172.0 mg, 0.8035 mmol), and potassium carbonate (278.30 mg, 2.0088 mmol) in water (0.9 mL) and 1,4-dioxane (3.0 ml), in a 48 mL sealed glass tube, was purged with nitrogen gas for 10 min. After adding palladium tetrakis (77.35 mg, 0.066 mmol), the reaction mixture was again purged with nitrogen gas for 30 minutes, and the mixture was heated at 80° C. for 16 h. The reaction progress was monitored by TLC (TLC silica gel plate, UV to visualize the spot) and LCMS. After completion of reaction, the mixture was cooled 25° C.-30° C. and filtered through a bed of celite and washed with ethyl acetate (40 mL). The combined organic layers were concentrated under reduced pressure to afford crude material (400 mg). The crude compound was then purified by Prep-HPLC. The collected fractions were concentrated under reduced pressure to afford Compound 1 (70 mg, 25.5%) as an off-white solid. TLC system: 10% MeOH in DCM (R_{f} value=0.3). ¹H NMR (400 MHz, DMSO-d6): δ8.54-8.52 (m, 1H), 8.33 (d, J=1.60 Hz, 1H), 8.02 (d, J=1.60 Hz, 1H), 7.99 (d, J=0.80 Hz, 1H), 7.84 (dd, J=8.00 Hz, 1H), 7.62-7.60 (m, 2H), 7.45 (dd, J=1.60, 8.00 Hz, 1H), 5.28 (s, 1H), 4.81 (d, J=3.60 Hz, 1H), 3.99 (br s, 1H), 3.78-3.73 (m, 1H), 3.51 (d, J=4.00 Hz, 1H), 3.30-3.15 (br s, 2H), 1.85-1.70 (m, 2H), 1.48 (s, 6H), 1.45-1.30 (m, 2H). LCMS: 97.32% (m/z=457.00[M+H]).

### Synthesis Example 2: Scaled Synthesis of Compound 1

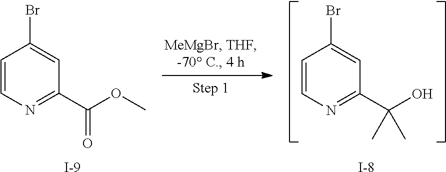

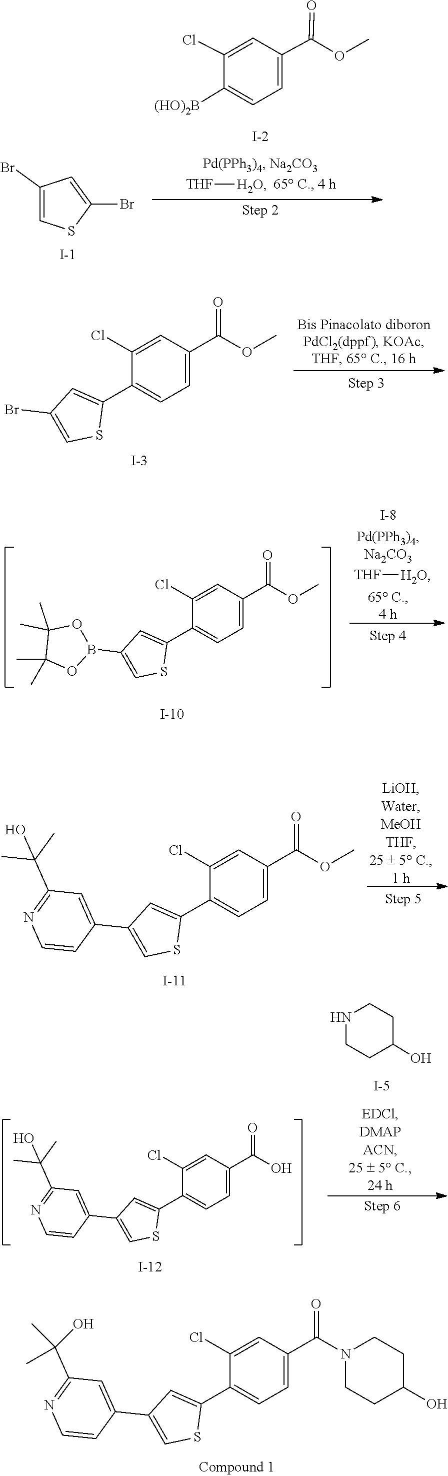

Compound 1 was alternatively synthesized according to the above scheme.

Step 1: Synthesis of 2-(4-bromopyridin-2-yl)propan-2-ol (I-8): To a stirred solution of Compound I-9 (200 g, 0.9258 mole) in tetrahydrofuran (3000 mL) was added methyl magnesium bromide solution (3.0 M in diethyl ether) (1543 mL, 4.628 mole) at −70±10° C. under a nitrogen atmosphere. The reaction mass was maintained at −65±5° C. for 3 and then quenched with saturated ammonium chloride solution (2000 mL) (started quenching at −65±5° C. and slowly raised temperature to 25±5° C.). The organic layer was separated and the aqueous layer was re-extracted with ethyl acetate (2000 mL). The combined organic layers were washed with water (2000 mL) dried and concentrated at 40±5° C. under reduced pressure (vacuum 100-300 mbar) to yield crude compound 2-(4-bromopyridin-2-yl)propan-2-ol (Compound 1-9) as a brown liquid (197 g, crude) which was used in Step 4 without further column purification.

Step 2: Synthesis of 4-(4-bromothiophen-2-yl)-3-chlorobenzoate (I-3): To a stirred solution of Compound I-1 (400 g, 1.6533 mole) in tetrahydrofuran (3200 mL) was charged Compound I-2 (319 g 1.4878) and aqueous sodium carbonate solution (Note: 350.18 g of sodium carbonate was dissolved into 3200 mL of water and purged with nitrogen for 30 minutes) at 25±5° C. under nitrogen atmosphere. Again the reaction mass was purged with nitrogen for 60 minutes and tetrakis(triphenylphosphine)palladium(0) (76.38 g, 0.0661 mole) was added into the reaction. The reaction mass was heated to 65±5° C. for 20 hours, then cooled to 25±5° C. and the organic layer was separated. The aqueous layer was extracted with ethyl acetate (2000 mL×2) and the combined organic layers were washed with water (2000 mL×2) and concentrated at 40±5° C. under reduced pressure (vacuum 100-300 mbar) to yield crude product. The crude product was purified by silica gel column chromatography (Silica Gel 60-120 mesh, using 0-2% ethyl acetate in petroleum ether as an eluent). The appropriate fractions were collected and concentrated at 40±5° C. under reduced pressure (vacuum 100-300 mbar) to afford methyl 4-(4-bromothiophen-2-yl)-3-chlorobenzoate (Compound 1-3) as an off-white solid (265.0 g, 53.7% yield).

Step 3: Synthesis of methyl 3-chloro-4-(4-(4,4,5,5-tetramethyl-1,3,2-dioxaborolan-2-yl)thiophen-2-yl)benzoate (I-10): To a stirred solution of Compound 1-3 (250 g, 0.754 mole) in tetrahydrofuran (2500 mL) was added bis(pinacolato)diboron (230 g, 0.905 mole) and potassium acetate (223 g, 2.272 mole) at 25±5° C. under a nitrogen atmosphere. The reaction mass was further purged with nitrogen for 20-30 minutes at 25±5° C. and [1,1′-bis(diphenylphosphino)ferrocene]dichloropalladium(II) (27.7 g 0.0378 mole) was added. The reaction mass was heated to 65±5° C. for 27 hours then cooled 25±5° C., filtered through a celite bed and with ethyl acetate (1000 mL) washes. The combined organic layers were concentrated at 40±5° C. under reduced pressure (vacuum 100-300 mbar) to yield a crude product. Petroleum ether (1500 mL) was added and, after stirring for 30 minutes, decanted from the solids and evaporated completely at 40±5° C. under reduced pressure (vacuum 100-300 mbar) to give the crude methyl 3-chloro-4-(4-(4,4,5,5-tetramethyl-1,3,2-dioxaborolan-2-yl)thiophen-2-yl)benzoate (Compound I-10) as a brown gummy mass (285 g, crude) which was used for next step without further purification considered as 100% yield.

Step 4: Synthesis of methyl 3-chloro-4-(4-(2-(2-hydroxypropan-2-yl)pyridin-4-yl)thiophen-2-yl)benzoate (I-11): To a stirred solution of Compound I-8 (156.0 g, 0.7219 mole) in tetrahydrofuran (1248 mL) was charged Compound I-10 (281.7 g, 0.7439) and aqueous sodium carbonate solution (Note: 153.0 g of sodium carbonate was dissolved into 1248 mL of water and purged with nitrogen for 60 minutes) at 25±5° C. under nitrogen atmosphere. The reaction mass was further purged with nitrogen for 60 minutes and then tetrakis(triphenylphosphine)palladium(0) (33.39 g, 0.0289 mole) was added into the reaction. The reaction mass was heated to 65° C. for 3 hours, cooled 25±5° C. and the organic layer was separated. The aqueous layer was extracted with ethyl acetate (780 mL×2) and the combined organic layers were washed with water (780 mL×2) and concentrated at 40±5° C. under reduced pressure (vacuum 100-300 mbar) to yield crude product. The crude product was purified by silica gel column chromatography (Silica Gel 100-200 mesh, using 10-30% ethyl acetate in petroleum ether as an eluent to afford methyl 3-chloro-4-(4-(2-(2-hydroxypropan-2-yl)pyridin-4-yl)thiophen-2-yl)benzoate (Compound I-11) as an off-white solid (180.0 g, 64.2% yield).

Step 5: Synthesis of 3-chloro-4-(4-(2-(2-hydroxypropan-2-yl)pyridin-4-yl)thiophen-2-yl)benzoic acid (Compound I-12): To a stirred mixture of compound 7 (180 g, 0.4645 mole) in tetrahydrofuran (1800 mL) was added methanol (1080 mL) at 25±5° C. The reaction mass was cooled to 5° C. and lithium hydroxide monohydrate solution (48.8 g of Lithium hydroxide monohydrate was dissolved in 720 mL of water) was slowly added at 5±5° C. The reaction mass was warmed to 25° C. for 1 hour and then the organic solvents were distilled out at 40±5° C. under reduced pressure (vacuum 100-300 mbar). The residue was dissolved in water (1800 mL) at 25±5° C. and washed with ethyl acetate (1500 mL×3). The aqueous layer pH was adjusted to ˜5 using saturated citric acid solution (−60 mL). The resulting slurry mass was stirred for 1 hour at 25±5° C. and filtered; washed with water (1000 mL). The wet solid was further dried at 50±5° C. under reduced pressure (vacuum 10-30 mbar) to yield 3-chloro-4-(4-(2-(2-hydroxypropan-2-yl)pyridin-4-yl)thiophen-2-yl)benzoic acid (Compound I-12) as an off-white solid (150.0 g, 86.4% yield).

Step 6: Synthesis of (3-chloro-4-(4-(2-(2-hydroxypropan-2-yl)pyridin-4-yl)thiophen-2-yl)phenyl) (4-hydroxypiperidin-1-yl)methanone (Compound 1): To a stirred solution of Compound I-12 (150 g, 0.401 mole) in acetonitrile (1500 mL) was added N-(3-dimethylaminopropyl)-N′-ethylcarbodiimide hydrochloride (115.4 g, 0.602 mole) and 4dimethylaminopyridine (73.5 g, 0.602 mole) at 25±5° C. After stirring for 10-15 minutes, compound I-5 (40.52 g, 0.401 mole) was added and the reaction mixture was maintained at 25±5° C. for 27 hours. The reaction was then quenched with water (1500 mL) and the acetonitrile was distilled out at 45±5° C. under reduced pressure (vacuum 100-300 mbar). The product was then extracted with ethyl acetate (1500 mL), washed with water (1500 mL×5) and concentrated at 45±5° C. under reduced pressure (vacuum 100-300 mbar) to the give a crude product. This material was purified by silica gel column chromatography (Silica Gel 100-200 mesh, using 0-5% methanol in dichloromethane as an eluent). The appropriate fractions were collected and concentrated at 40±5° C. under reduced pressure (vacuum 100-300 mbar) to afford (3-chloro-4-(4-(2-(2-hydroxypropan-2-yl)pyridin-4-yl)thiophen-2-yl)phenyl) (4-hydroxypiperidin-1-yl)methanone (Compound 1) as an off-white solid (92.0 g, 50.2% yield). Characterization matches that of the corresponding step in Example 1.

### Biological Example 1: Reporter Screening Assay

The effect of Compound 1 on transcriptional activity SREBP was evaluated using an SRE-luciferase reporter construct. The EC50 of Compound 1 in HepG2 stably expressing LSS-Luciferase transgene was 150 nM.

After 24 hours incubation medium cells were treated with various Lovastatin concentrations (10 μM, 5 μM and 1 μM). After 24 hours treatment plates were covered with aluminum foil and frozen at −80° C. overnight. Plates were thawed to room temperature and Luciferase assay was performed. Cells were seeded in a 96 well plate in 10% FCS DMEM media and incubated for 24 hours. Then, they are switched to 0% FCS Media with Compound 1 or DMSO control for another 24 hours, at which point they are assayed for activity of LSS-reporter.

Reagents for performing Luciferase assay were stored at −20° C. To a tube of lyophilized assay substrate was added 1 mL Substrate Solvent and mixed well. The Substrate tube after reconstitution was covered with aluminum foil so as to keep it protected from light. The assay buffer was thawed to room temperature. To 20 mL Assay Buffer was added 200 μL of reconstituted 100× Substrate and mixed well. The reconstituted substrate as well as the assay solution (buffer+substrate) was protected from light throughout the procedure by keeping it covered with aluminum foil.

Using a multi-channel pipette, 100 μL Assay Solution (buffer+substrate) was added directly to each sample well in Plate 1, which was incubated for 30 min (plate was covered with aluminum foil). After 30 min incubation, the plate was read for total luminescence. Each well was read for 2 seconds in a plate luminometer. (Microplate reader Envision Microplate reader from Perkin Elmer). Precaution was taken to incubate plate exactly for 30 min prior to reading on the plate reader.

Materials: SREBPv1 Reporter cell line: HepG2-#32251. Growth Medium: MEM (Corning 10-010), 10% FBS, 1% GlutaMax (Invitrogen Catalog #35050061), μg/ml Puromycin (Invitrogen Catalog #A1113803) and 1% Penicillin-Streptomycin (Pen-Strep). Treatment Media: Phenol-free MEM (Invitrogen Catalog #51200-038) and 1% GlutaMax (Invitrogen Catalog #35050061). Luciferase Assay: LightSwitch Luciferase Assay Kit (Catalog #32032). LDH assay: Pierce LDH Cytotoxicity Assay Kit (Catalog#SD249616).

### Biological Example 2: Gene Expression Testing

The effect of Compound 1 on the gene expression of HepG2 cells was evaluated.

HepG2 cells (P2) were seeded in 24 well plate (80,000 cells/ well) for RNA extraction and in a 96 well plate (10,000 cells/ well) for Cell Titer Glow (CTG). The media used was DMEM and contained 10% FBS. Compound 1 was evaluated at 500 nm for 48 hours. Two biological replicates per experimental group were evaluated for RNA, and 3 biological replicates per experimental group analyzed with CTG.

For gene analysis RNA was harvested with RNEasy kit and 20-100 ng used to synthesize cDNA with random primers. Quantitative PCR was performed on 1 pg to 100 ng cDNA for the following genes: ACACA, ACLY, FASN, LSS, PNPLA3. Gene expression levels were determined using MET method comparing treated to mock or vehicle treated cells as a baseline. The results are presented in Table 1 below.

TABLE 1
Q-PCR analysis of SREBP Target Gene in HepG3 cells treated
with Compound 1for 48 hour, 500 nM

| | | | | | | Total |
|---|---|---|---|---|---|---|
| | | | | | | Gene |
| | ACACA | ACLY | FASN | LSS | PNPLA3 | Average |
| Compound | 0.648 | 0.663 | 0.542 | 0.295 | 0.187 | 0.467 |
| 1 | | | | | | |

### Biological Example 3: Additional Gene Expression Testing

Quantitative PCR will also be performed for the following additional genes, according to the procedure described in Biological Example 2: HMGCR, MVD, MVK, ACSS1, ACSS2, ACACB, ELOVL6, SCD, SREBF1, SREBF2, SCAP, ACTB18S.

### Biological Example 4: Evaluation of in vitro ADME properties of Compound 1

The ADME properties of Compound 1 in vitro were evaluated. Results are presented in Table 2.

TABLE 2
In vitro ADME properties

| | Kinetic | Human | Mouse | Rat | | |
|---|---|---|---|---|---|---|
| | Solubility: | LM: | LM: | LM: | | In Vitro % |
| | Mean | Half | Half | Half | | Fu |
| | Solubility | life | life | life | Log | (Hu/Mo |
| | (uM) | (Min) | (Min) | (Min) | D | Plasma) |
| Com- | 37 | 64.7 | 120 | 101 | 3.38 | 2.6/0.7 |
| pound 1 | | | | | | |

Kinetic Solubility Procedure: A 10 mM stock solution of Compound 1 was prepared in DMSO, then 4 μL of the stock was added to a deep well plate containing 396 μL of pH 7.4 buffer. The sample plate was vortexed at 800 rpm for 24 h on thermomixer at room temperature. The plate was sealed well during the incubation process. The DMSO content in the sample was 1.0%. The concentration of Compound 1 in the final incubation was 100 μM. At the end of the incubation period, the sample plate was centrifuged at 4000 rpm for 10 min and analyzed in LC-UV against a calibration curve (CC).

Half-life Human Microsomes: Compound 1 was evaluated for stability in human liver microsomes. A 10 mM stock solution of Compound 1 was prepared in DMSO and diluted with water: acetonitrile (1:1) to a concentration of 1 mM. A working concentration of 100 μM was prepared by further dilution with water: acetonitrile (1:1). To make the preincubation mixture, 2.5 μL of the diluted Compound 1 was combined with 75 μL of human liver microsomes at 3.33 mg/mL, and 85 μL of 100 mM potassium phosphate buffer, and this mixture pre-incubated for 10 min at 37° C. To make the 60 minute mixture without cofactor, 32.5 μL of the preincubation mixture was combined with 17.5 μL of 100 mM potassium phosphate buffer and incubated for 60 min at 37° C. To make the 0 min sample with cofactor (NADPH), 16.25 μL of the preincubation mixture was combined with 200 μL of acetonitrile containing internal standard and 8.75 μL of cofactor (NADPH). To make the incubation mixture, 62 μL of cofactor (2.85 mM) was combined with the remaining incubation mixture, and incubated for 60 min at 37° C. To prepare the sample mixture to be evaluated, 25 μL incubation mixture was combined with 200 μL of acetonitrile containing internal standard and vortexed for 5 min at 1200 rpm, then centrifuged for 10 min at 4000 rpm. The supernatant was diluted 2 fold with water and injected on LC-MS/MS. The sample mixture was evaluated by LC-MS/MS using 10 mM ammonium acetate with 0.1% FA as the aqueous mobile phase, and methanol as the organic mobile phase.

Half-life Rat and Mouse Microsomes: Compound 1 was evaluated in rat and mouse liver microsomes following a similar procedure as described above for human liver microsomes.

Log D Procedure: The Log D of Compound 1 was evaluated by octanol/aqueous buffer partitioning. 500 μL of organic phase (1-octanol) was added to each well of a 2 mL deep well plate, followed by 500 μL of buffer and 15 μL of test compound in DMSO (0.15 mM). The plate was vortexed for 10 seconds and incubated at room temperature for 1 hr on a plate shaker at 200 rpm. After incubation, the samples were allowed to equilibrate for 20 min and then centrifuged at 4000 rpm for 30 min for complete phase separation. The distribution of test compound in buffer and octanol phase was analyzed by HPLC-UV. Log D=Log (Area of Octanol/Area of Buffer)

### Biological Example 5: Evaluation of In Vivo Pharmacokinetic Properties of Compound 1

The in vivo pharmacokinetic properties of Compound 1 by both intravenous and oral administration were evaluated in male C57 BL/6 mice. The in vivo pharmacokinetic properties of Compound 2 by both intravenous and oral administration were evaluated in male C57 BL/6 mice for comparison.

Animals were housed in cages with clean bedding. Certified rodent diet was provided. Water was available ad libitum. Environmental controls for the animal room were set to maintain a temperature of 22 to 25° C., humidity of 40-70% RH, and a 12-hour light /12-hour dark cycle. Normal healthy animals certified by the attending veterinarian were selected and acclimatized for minimum three days prior to initiation of study.

Procedure for Blood Withdrawal: Mice were anesthetized using gaseous anesthesia. Blood samples were collected through a capillary, guided in retro-orbital plexus. At each time point, blood samples from 3 mice of each respective group were collected. The blood samples were collected in pre-labeled tubes. 0.2 to 0.3 mL of blood were collected from each mouse. After collection of the blood samples at each time point, the samples were stored on ice prior to centrifugation. Blood samples were centrifuged within 15 minutes to separate plasma. Centrifugations was done at 1540 rcf (5000 rpm) at 4° C. for 10 minutes. The plasma was separated and transferred to pre-labeled micro-centrifuge tubes and promptly frozen at −80±10° C. until bioanalysis was performed. Samples were identified by test item, group, animal number, and collection time point.

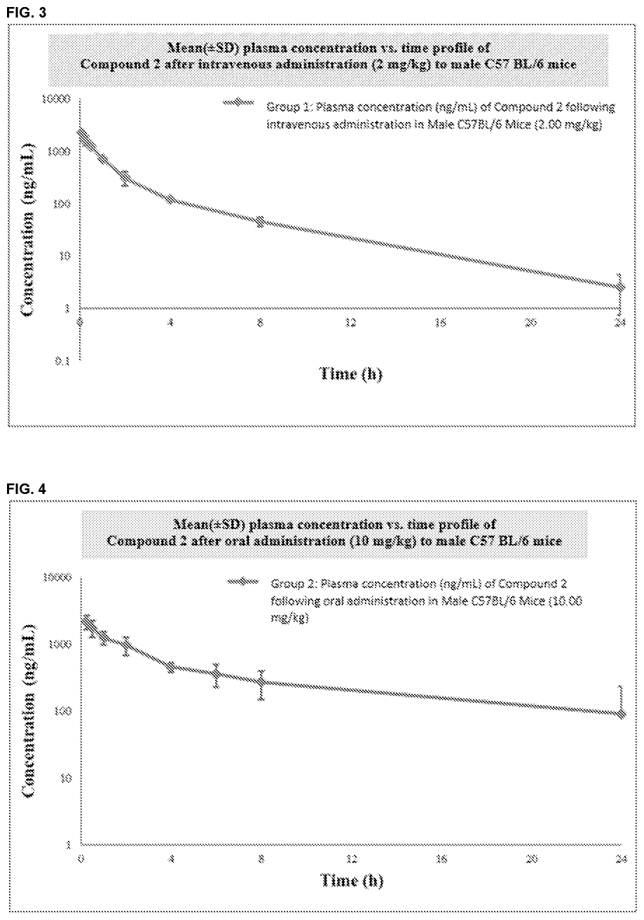

To evaluate pharmacokinetic properties of intravenous delivery, nine male C57 BL/6 mice were administered 2.00 mg Compound 1/kg animal weight through the tail vein. The concentration of Compound 1 in the plasma of the animals was evaluated at 0.083, 0.25, 0.5, 1, 2, 4, 8, and 24 hr by taking blood samples from the mice. A graph of the plasma concentration vs. time is provided in FIG. 1. A summary of the pharmacokinetic parameters of intravenous delivery of 2.00 mg Compound 1/kg animal weight is provided in Table 3. Compound 2 was similarly administered for comparison. A graph of the plasma concentration vs. time is provided in Table 3. A summary of the pharmacokinetic parameters of intravenous delivery of 2.00 mg Compound 2/kg animal weight is provided in FIG. 3.

TABLE 3
In vivo pharmacokinetic properties (intravenous)

| | Compound 1 | Compound 2 |
|---|---|---|
| Dose (mg/kg) | 2.00 | 2.43 |
| _{C0} (ng/mL) | 4632.11 | 2672.26 |
| T1/2 (h) | 6.50 | 3.67 |
| AUC₀₋ₗₐₛₜ (ng · h/mL) | 23838.92 | 2810.32 |
| AUC_{0-inf} (ng · h/mL) | 25708.44 | 2823.89 |
| AUC_{Extra}(%) | 7.27 | 0.48 |
| Cl (ml/min/kg) | 1.30 | 14.34 |
| Vd (L/kg) | 0.73 | 4.55 |
| Vss (L/kg) | 0.70 | 2.32 |
| MRT₀₋ₗₐₛₜ (h) | 7.07 | 2.56 |

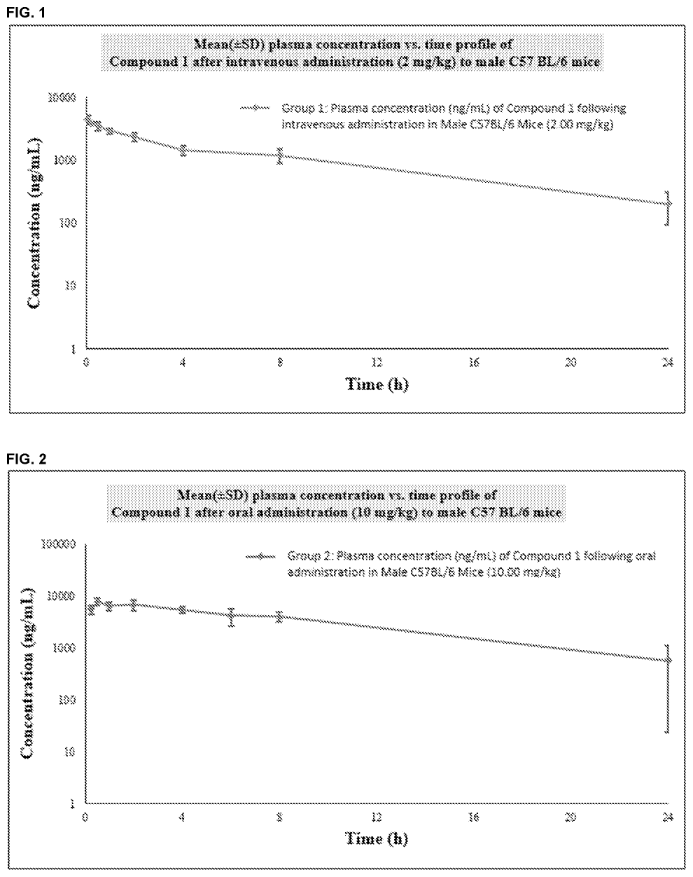

To evaluate pharmacokinetic properties of oral delivery, nine male C57 BL/6 mice were administered 10 mg Compound 1/kg animal weight by mouth. The concentration of Compound 1 in the plasma of the animals was evaluated at 0.25, 0.5, 1, 2, 4, 6, 8, and 24 hr by taking blood samples. A graph of the plasma concentration vs. time is provided in FIG. 2. A summary of the pharmacokinetic parameters of oral delivery of 10 mg Compound 1/kg animal weight is provided in Table 4. Compound 2 was similarly administered for comparison. A graph of the plasma concentration vs. time is provided in Table 4. A summary of the pharmacokinetic parameters of oral delivery of 10 mg Compound 2/kg animal weight is provided in FIG. 4.

TABLE 4
In vivo pharmacokinetic properties (oral)

| | Compound 1 | Compound 2 |
|---|---|---|
| Dose (mg/kg) | 10.00 | 12.01 |
| Cmax(ng/mL) | 7838.11 | 2176.93 |
| Tmax (hr) | 0.50 | 0.25 |
| t1/2 (h) | 6.18 | 9.46 |
| AUC₀₋ₗₐₛₜ (ng · h/mL) | 71174.48 | 8059.89 |
| AUC_{0-inf} (ng · h/mL) | 76345.99 | 9304.90 |
| AUC_{Extra}(%) | 6.77 | 13.38 |
| MRT₀₋ₗₐₛₜ (h) | 7.60 | 6.61 |
| Rsq | 0.9951 | 0.9906 |
| % F | 59.71 | 66.64 |

### Biological Example 6: Evaluation of In Vivo Pharmacology of Compound 1 using C33A Endometrial Cell Line

Animals were housed in cages with clean bedding. Certified rodent diet and water were available ad libitum. Environmental controls for the animal room were set to maintain a temperature of 22 to 25° C., humidity of 40-70% RH, and a 12-hour light /12-hour dark cycle. Normal healthy animals certified by the attending veterinarian were selected and acclimatized for minimum three days prior to initiation of study.

Balb/c nude mice (age 6-8 weeks) were kept in Individually Ventilated Cages with a maximum of 5 animals in each cage. The bedding material (corn cob) was changed twice per week.

C33A tumor cells were cultured in Eagle's Minimum Essential medium, supplemented with 10% heat inactivated fetal bovine serum, 100U/mL penicillin, and 100 μg/ml streptomycin at 37° C. in an atmosphere of 5% CO₂ in air. The tumor cells were routinely subcultured 2-3 times weekly. The cells were harvested during an exponential growth phase and were counted for tumor inoculation.

Each Balb/c nude mouse was inoculated subcutaneously at the right flank with the C33A tumor cells (1e6) in 0.1 mL PBS for tumor development.

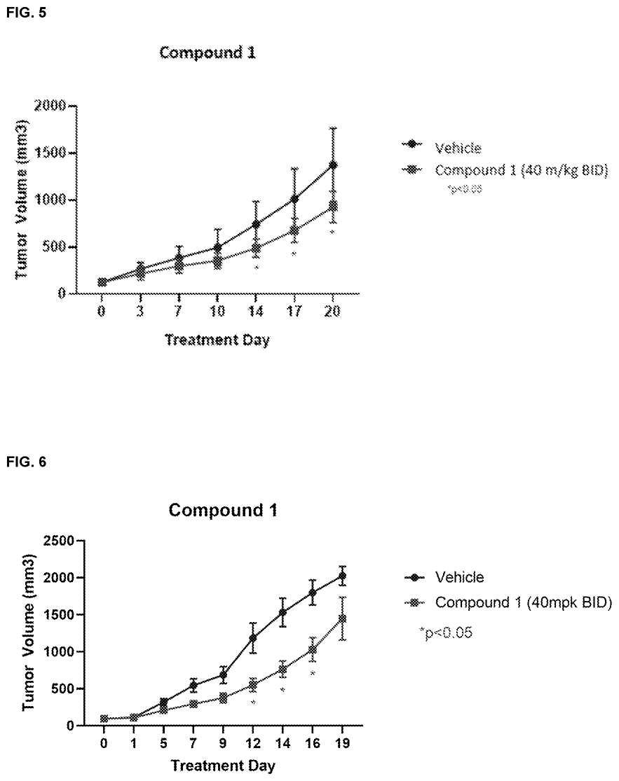

Tumor volume was measured twice weekly, up to 20 days, in two dimensions using a caliper, and the volume was expressed in mm³ using the formula V=0.5 a×b² where a and b are the long and short diameters of the tumor, respectively. A graph showing the effect of Compound 1 on tumor volume compared to that of vehicle is provided in FIG. 5.

### Biological Example 7: Evaluation of In Vivo Pharmacology of Compound 1 using A2780 Ovarian Carcinoma Cell Line

Female mice (Mus Musculus, strain Foxn1^{nu/nu}, 6-8 weeks old, 18-22 g) were supplied from Shanghai Lingchang Bio-Technology Co., Ltd. The mice were kept in Individual Ventilation Cages at constant temperature (20-26° C.) and humidity (40-70%) with 3 animals in each cage. Animals had free access to sterile drinking water and irradiation-sterilized dry granule food during the entire study period (Jiangsu Province Synergy Pharmaceutical Bioengineering Co., Ltd. Cat #1010019). Animals were marked by ear coding (10 animals per group).

Cell Culture: A2780 tumor cells were cultured in EMEM (Eagle's Minimum Essential Medium) supplemented with 10% heat inactivated fetal bovine serum, 100U/mL penicillin and 100 μg/mL streptomycin at 37° C. in an atmosphere of 5% CO₂ in air. The tumor cells were routinely sub-cultured two to three times weekly. The cells growing in an exponential growth phase were harvested and counted for tumor inoculation. Cells with greater than 90% viability were used for tumor inoculation. Mycoplasma testing was performed weekly during culture and STR testing was done to verify cell line.

Tumor Inoculation and Measurement: Each mouse was inoculated subcutaneously on the right flank with 1×10⁶ A2780 tumor cells (live viable cells) per animal in a volume of 0.1 mL PBS with/without 50% Matrigel® (Corning, cat#: 354234) to aid tumor development. 26 gauge needles were used for the injection. After tumors were palpable, tumor volume was measured twice weekly in two dimensions using a caliper, and the volume expressed in mm³ using the formula: V=0.5 a×b², where a and b are the long and short diameters of the tumor, respectively. Tumor size was measured twice weekly until tumor size reached endpoint size (2000 mm³) and animals were humanely euthanized or the animals had been dosed for 21 days and animals euthanized and samples collected for bioanalysis. A graph showing the effect of Compound 1 on tumor volume compared to that of vehicle is provided in FIG. 6.

### Biological Example 8: Evaluation of Compound 1 on Cancer Cell Growth Inhibition

The ability of select compounds to inhibit cell growth in various cancer cell lines are evaluated. Cells are treated with compound at doses ranging from 10 uM to 1 pM to generate IC₅₀ curves of cell line growth inhibition. The cell lines shown in Table 5 have reduced growth by at least 50% at 10 uM or lower over 72 hr treatment in growth media. The cell lines shown in Table 6 have reduced growth by at least 50% with an IC₅₀ greater than 10 uM.

Six 10-fold compound dilutions are prepared in DMSO (e.g. 10 mM, 1 mM, 100 uM, 10 uM, 1 uM, and 0.1 uM). Single data points are acquired for each concentration. The final concentration of DMSO is 0.1%. The duration of the treatment is 72 hr. Growth inhibition is measured using Sulforhodamine B in a protein staining assay. Activity of the agents is determined by evaluation the following parameters: IC₅₀, GI₅₀, IC₁₀, TGI, LC₅₀, IC₉₀, and GI₉₀ (where these values can be calculated).

TABLE 5
Responder Cell Lines with IC50 < 10 uM
Compound 1

| Cell Line | Tissue of Origin |
|---|---|
| PLCPRF5 | liver |
| C33A | endometrial |
| A2780 | ovary |
| SKHEP1 | liver |
| SKMEL5 | skin |
| CACO2 | colon |
| K-562 | hematological |
| L-363 | hematological |
| ACHN | kidney |
| MDAMB468 | breast |
| MCF7 | breast |
| HEPG2 | liver |
| NCIH82 | lung |
| HT1080 | connective tissue |
| OVCAR3 | ovary |
| JEG3 | placenta |
| MT3 | breast |
| JIMT1 | breast |
| SU-DHL-6 | hematological |
| MV4-11 | hematological |
| HCT116 | colon |
| JAR | placenta |
| ASPC1 | pancreas |
| WSU-NHL | hematological |
| A549 | lung |
| 22RV1 | prostate |
| HEK293 | kidney |
| MINO | hematological |
| HCT15 | colon |
| SF295 | brain |
| SKNSH | brain |
| LOVO | colon |
| RAMOS | hematological |
| MHHESI | bone |
| HL-60 | hematological |
| TE671 | muscle |
| A204 | muscle |
| MDAMB435 | skin |
| THP-1 | hematological |
| PANC1005 | pancreas |
| COLO205 | colon |
| EFO21 | ovary |
| SKBR3 | breast |

TABLE 6
Non-Responder Cell Lines with IC50 >10 uM
or Not Determined
Compound 1

| Cell Line | Tissue of Origin |
|---|---|
| DLD1 | colon |
| CALU6 | lung |
| SU-DHL-10 | hematological |
| OVCAR4 | ovary |
| KASUMI-1 | hematological |
| BT20 | breast |
| MDAMB436 | breast |
| MDAMB468 | breast |
| MG63 | bone |
| MHHES1 | bone |
| MIAPACA2 | pancreas |
| MINO | hematological |
| MT3 | breast |
| MV4-11 | hematological |
| NCIH292 | lung |
| NCIH358M | lung |
| NCIH460 | lung |
| NCIH82 | lung |
| OVCAR3 | ovary |
| OVCAR4 | ovary |
| PANC1 | pancreas |
| PANC1005 | pancreas |
| PBMC | hematological |
| PC3 | prostate |
| PLCPRF5 | liver |
| RAMOS | hematological |
| RD | muscle |
| RDES | bone |
| SAOS2 | bone |
| SF268 | brain |
| SF295 | brain |
| SKBR3 | breast |
| SKHEP1 | liver |
| SKLMS1 | uterus |
| SKMEL28 | skin |
| SKMEL5 | skin |
| SKNAS | brain |
| SKNSH | brain |
| SKOV3 | ovary |
| SNB75 | brain |
| SU-DHL-10 | hematological |
| SU-DHL-6 | hematological |
| SW620 | colon |
| T24 | bladder |
| TE671 | muscle |
| THP-1 | hematological |
| U2OS | bone |
| U87MG | brain |
| UMUC3 | bladder |
| UO31 | kidney |
| WSU-NHL | hematological |

1. The compound (3-chloro-4-(4-(2-(2-hydroxypropan-2-yl)pyridin-4-yl)thiophen-2-yl)phenyl)(4-hydroxypiperidin-1-yl)methanone: 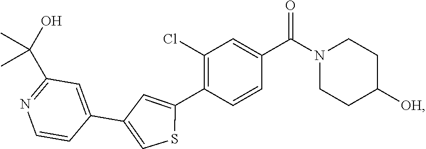 [SMILES: CC(C)(O)c1cc(-c2csc(-c3ccc(C(=O)N4CCC(O)CC4)cc3Cl)c2)ccn1]
or a pharmaceutically acceptable salt, solvate, tautomer, isotope, or isomer thereof.

2. A pharmaceutical composition, comprising the compound claim 1, or a pharmaceutically acceptable salt, solvate, tautomer, isotope, or isomer thereof, and a pharmaceutically acceptable excipient.

3. A method of inhibiting a sterol regulatory element-binding protein (SREBP), comprising contacting the SREBP or contacting an SREBP cleavage activating-protein (SCAP) with the compound of claim 1, or a pharmaceutically acceptable salt, solvate, tautomer, isotope, or isomer thereof.

4. A method of inhibiting the proteolytic activation of a sterol regulatory element-binding protein (SREBP), comprising contacting an SREBP cleavage activating-protein (SCAP) with the compound of claim 1, or a pharmaceutically acceptable salt, solvate, tautomer, isotope, or isomer thereof.

5. A method of treating a disorder in a subject in need thereof, wherein the disorder is mediated by a sterol regulatory element-binding protein (SREBP), comprising administering to the subject an effective amount of the compound of claim 1, or a pharmaceutically acceptable salt, solvate, tautomer, isotope, or isomer thereof.

6. A method of treating a disorder in a subject in need thereof, comprising administering to the subject an effective amount of the compound of claim 1, or a pharmaceutically acceptable salt, solvate, tautomer, isotope, or isomer thereof.

7.-12 (canceled)

13. The method of claim 3, comprising contacting SREBP or SCAP with the compound, or pharmaceutically acceptable salt, solvate, tautomer, isotope, or isomer thereof, wherein the expression of one or more genes selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SREBP or SCAP with the compound, or pharmaceutically acceptable salt, solvate, tautomer, isotope, or isomer thereof.

14. The method of claim 6, wherein the disorder is Metabolic Syndrome, type 2 diabetes, obesity, liver disease, insulin resistance, adiposopathy, or dyslipidemia.

15. The method of claim 14, wherein the dyslipidemia is hypertriglyceridemia or elevated cholesterol levels.

16. The method of claim 14, wherein the liver disease is nonalcoholic steatohepatitis, liver fibrosis, or liver inflammation, or a combination thereof.

17. The method of claim 6, wherein the disorder is a hyperproliferative disorder.

18. The method of claim 17, wherein the hyperproliferative disorder is cancer.

19. The method of claim 18, wherein the cancer is breast cancer, liver cancer, ovarian cancer, pancreatic cancer, prostate cancer, a soft tissue sarcoma, bladder cancer, endometrial cancer, skin cancer, colon cancer, hematologic cancer, placenta cancer, brain cancer, kidney cancer, lung cancer, or bone cancer.

20. The method of claim 6, wherein the disorder is endotoxic shock, systemic inflammation, or atherosclerosis.

21.-60. (canceled)

61. A method of treating non-alcoholic steatohepatitis (NASH) in a subject in need thereof, comprising administering to the subject an effective amount of the compound of claim 1, or a pharmaceutically acceptable salt, solvate, tautomer, isotope, or isomer thereof.

62.-63. (canceled)

64. A method of treating a hyperproliferative disorder in a subject in need thereof, comprising administering to the subject an effective amount of the compound of claim 1, or a pharmaceutically acceptable salt, solvate, tautomer, isotope, or isomer thereof.

65.-72. (canceled)

73. The method of claim 4, comprising contacting SCAP with the compound, or pharmaceutically acceptable salt, solvate, tautomer, isotope, or isomer thereof, wherein the expression of one or more genes is selected from the group consisting of ACSS2, ALDOC, CYP51A1, DHCR7, ELOVL6, FASN, FDFT1, FDPS, HMGCS1, HSD17B7, IDI1, INSIG1, LDLR, LSS, ME1, PCSK9, PMVK, RDH11, SC5DL, SQLE, STARD4, TM7SF2, PNPLA3, SREBF1, SREBF2, HMGCR, MVD, MVK, ACLY, MSMO1, ACACA, and ACACB is reduced after contacting the SCAP with the compound, or pharmaceutically acceptable salt, solvate, tautomer, isotope, or isomer thereof.

74. The method of claim 5, wherein the disorder is Metabolic Syndrome, type 2 diabetes, obesity, liver disease, insulin resistance, adiposopathy, or dyslipidemia.

75. The method of claim 74, wherein the dyslipidemia is hypertriglyceridemia or elevated cholesterol levels.

76. The method of claim 74, wherein the liver disease is nonalcoholic steatohepatitis, liver fibrosis, or liver inflammation, or a combination thereof.

77. The method of claim 5, wherein the disorder is a hyperproliferative disorder.

78. The method of claim 77, wherein the hyperproliferative disorder is cancer.

79. The method of claim 78, wherein the cancer is breast cancer, liver cancer, ovarian cancer, pancreatic cancer, prostate cancer, a soft tissue sarcoma, bladder cancer, endometrial cancer, skin cancer, colon cancer, hematologic cancer, placenta cancer, brain cancer, kidney cancer, lung cancer, or bone cancer.

80. The method of claim 64, wherein the hyperproliferative disorder is cancer.

81. The method of claim 80, wherein the cancer is breast cancer, liver cancer, ovarian cancer, pancreatic cancer, prostate cancer, a soft tissue sarcoma, bladder cancer, endometrial cancer, skin cancer, colon cancer, hematologic cancer, placenta cancer, brain cancer, kidney cancer, lung cancer, or bone cancer.
